(19) United States
(12) Patent Application Publication
(10) Pub. No.: US 2022/0233820 A1
(43) Pub. Date: Jul. 28, 2022

(54) SYSTEMS, APPARATUS AND METHODS FOR SUPPORTING AND DRIVING ELONGATED MEDICAL DEVICES IN A ROBOTIC CATHETER-BASED PROCEDURE SYSTEM

(71) Applicant: Corindus, Inc., Waltham, MA (US)

(72) Inventors: Andrew Clark, Waltham, MA (US); Eric Klem, Lexington, MA (US); Omid Saber, Waltham, MA (US); Saeed Sokhanvar, Medfield, MA (US); Peter Falb, Hingham, MA (US); Cameron Canale, Groton, MA (US); Paul Gregory, Watertown, MA (US)

(21) Appl. No.: 17/597,044

(22) PCT Filed: Jul. 14, 2020
(86) PCT No.: PCT/US20/41960

§ 371 Date: Dec. 23, 2021

Related U.S. Application Data
(60) Provisional application No. 62/874,222, filed on Jul. 15, 2019.

(51) Int. Cl. A61M 25/01; A61M 25/10; A61M 25/09

(52) U.S. Cl. CPC A61M 25/0113; A61M 25/10; A61M 39/06; A61M 2025/0183; A61M 25/09041

(57) ABSTRACT

An apparatus for providing support to an elongated medical device is provided between a first device module and a second device module coupled to a linear member of a robotic drive for a catheter-based procedure system. The second device module is located in a position along the linear member distal to the first device module. The apparatus includes a device support having a distal end and a proximal end. A section of the device support is positioned within the first device module. The apparatus also includes a connector attached to the distal end of the device support. The connector includes an attachment mechanism for engaging a proximal end of the second device module. The proximal end of the device support is configured to be coupled to the second device module.

### CROSS-REFERENCE TO RELATED APPLICATIONS

This application is based on, claims priority to, and incorporates herein by reference in its entirety U.S. Ser. No. 62/874,222, filed Jul. 15, 2019, and entitled “Systems, Apparatus and Methods for Supporting and Driving Elongated Medical Devices in a Robotic Catheter-Based Procedure System.”

### FIELD

The present invention relates generally to the field of robotic medical procedure systems and, in particular, to systems, apparatus and methods for supporting and driving elongated medical devices in a robotically controlled interventional procedure using a catheter-based procedure system.

### BACKGROUND

Catheters and other elongated medical devices (EMDs) may be used for minimally invasive medical procedures for the diagnosis and treatment of diseases of various vascular systems, including neurovascular intervention (NVI) also known as neurointerventional surgery, percutaneous coronary intervention (PCI) and peripheral vascular intervention (PVI). These procedures typically involve navigating a guidewire through the vasculature, and via the guidewire advancing a catheter to deliver therapy. The catheterization procedure starts by gaining access into the appropriate vessel, such as an artery or vein, with an introducer sheath using standard percutaneous techniques. Through the introducer sheath, a sheath or guide catheter is then advanced over a diagnostic guidewire to a primary location such as an internal carotid artery for NVI, a coronary ostium for PCI, or a superficial femoral artery for PVI. A guidewire suitable for the vasculature is then navigated through the sheath or guide catheter to a target location in the vasculature. In certain situations, such as in tortuous anatomy, a support catheter or microcatheter is inserted over the guidewire to assist in navigating the guidewire. The physician or operator may use an imaging system (e.g., fluoroscope) to obtain a cine with a contrast injection and select a fixed frame for use as a roadmap to navigate the guidewire or catheter to the target location, for example, a lesion. Contrast-enhanced images are also obtained while the physician delivers the guidewire or catheter so that the physician can verify that the device is moving along the correct path to the target location. While observing the anatomy using fluoroscopy, the physician manipulates the proximal end of the guidewire or catheter to direct the distal tip into the appropriate vessels toward the lesion or target anatomical location and avoid advancing into side branches.

Robotic catheter-based procedure systems have been developed that may be used to aid a physician in performing catheterization procedures such as, for example, NVI, PCI and PVI. Examples of NVI procedures include coil embolization of aneurysms, liquid embolization of arteriovenous malformations and mechanical thrombectomy of large vessel occlusions in the setting of acute ischemic stroke. In an NVI procedure, the physician uses a robotic system to gain target lesion access by controlling the manipulation of a neurovascular guidewire and microcatheter to deliver the therapy to restore normal blood flow. Target access is enabled by the sheath or guide catheter but may also require an intermediate catheter for more distal territory or to provide adequate support for the microcatheter and guidewire. The distal tip of a guidewire is navigated into, or past, the lesion depending on the type of lesion and treatment. For treating aneurysms, the microcatheter is advanced into the lesion and the guidewire is removed and several embolization coils are deployed into the aneurysm through the microcatheter and used to block blood flow into the aneurysm. For treating arteriovenous malformations, a liquid embolic is injected into the malformation via a microcatheter. Mechanical thrombectomy to treat vessel occlusions can be achieved either through aspiration and/or use of a stent retriever. Depending on the location of the clot, aspiration is either done through an aspiration catheter, or through a microcatheter for smaller arteries. Once the aspiration catheter is at the lesion, negative pressure is applied to remove the clot through the catheter. Alternatively, the clot can be removed by deploying a stent retriever through the microcatheter. Once the clot has integrated into the stent retriever, the clot is retrieved by retracting the stent retriever and microcatheter (or intermediate catheter) into the guide catheter.

In PCI, the physician uses a robotic system to gain lesion access by manipulating a coronary guidewire to deliver the therapy and restore normal blood flow. The access is enabled by seating a guide catheter in a coronary ostium. The distal tip of the guidewire is navigated past the lesion and, for complex anatomies, a microcatheter may be used to provide adequate support for the guidewire. The blood flow is restored by delivering and deploying a stent or balloon at the lesion. The lesion may need preparation prior to stenting, by either delivering a balloon for pre-dilation of the lesion, or by performing atherectomy using, for example, a laser or rotational atherectomy catheter and a balloon over the guidewire. Diagnostic imaging and physiological measurements may be performed to determine appropriate therapy by using imaging catheters or fractional flow reserve (FFR) measurements.

In PVI, the physician uses a robotic system to deliver the therapy and restore blood flow with techniques similar to NVI. The distal tip of the guidewire is navigated past the lesion and a microcatheter may be used to provide adequate support for the guidewire for complex anatomies. The blood flow is restored by delivering and deploying a stent or balloon to the lesion. As with PCI, lesion preparation and diagnostic imaging may be used as well.

When support at the distal end of a catheter or guidewire is needed, for example, to navigate tortuous or calcified vasculature, to reach distal anatomical locations, or to cross hard lesions, an over-the-wire (OTW) catheter or coaxial system is used. An OTW catheter has a lumen for the guidewire that extends the full length of the catheter. This provides a relatively stable system because the guidewire is supported along the whole length. This system, however, has some disadvantages, including higher friction, and longer overall length compared to rapid-exchange catheters (see below). Typically to remove or exchange an OTW catheter while maintaining the position of the indwelling guidewire, the exposed length (outside of the patient) of guidewire must be longer than the OTW catheter. A 300 cm long guidewire is typically sufficient for this purpose and is often referred to as an exchange length guidewire. Due to the length of the guidewire, two operators are needed to remove or exchange an OTW catheter. This becomes even more challenging if a triple coaxial, known in the art as a tri-axial system, is used (quadruple coaxial catheters have also been known to be used). However, due to its stability, an OTW system is often used in NVI and PVI procedures. On the other hand, PCI procedures often use rapid exchange (or monorail) catheters. The guidewire lumen in a rapid exchange catheter runs only through a distal section of the catheter, called the monorail or rapid exchange (RX) section. With a RX system, the operator manipulates the interventional devices parallel to each other (as opposed to with an OTW system, in which the devices are manipulated in a serial configuration), and the exposed length of guidewire only needs to be slightly longer than the RX section of the catheter. A rapid exchange length guidewire is typically 180-200 cm long. Given the shorter length guidewire and monorail, RX catheters can be exchanged by a single operator. However, RX catheters are often inadequate when more distal support is needed.

### SUMMARY

In accordance with an embodiment, an apparatus for providing support to an elongated medical device between a first device module and a second device module coupled to a linear member of a robotic drive for a catheter. The second device module is located in a position along the linear member distal to the first device module. The apparatus incudes a device support having a distal end and a proximal end. A section of the device support is positioned within the first device module. The apparatus also includes a connector attached to the distal end of the device support. The connector including an attachment mechanism for engaging a proximal end of the second device module. The proximal end of the device support is configured to be coupled to the second device module.

In accordance with another embodiment, a cassette for use in a robotic drive of a catheter-based procedure system includes a housing having a distal end and a proximal end, a device support having a lengthwise slit, a distal end and a proximal end, a connector attached to a distal end of the device support and a splitter positioned at the distal end of the housing of the cassette at an entry point for an elongated medical device into the device support. A section of the device support is positioned within the housing. In a first position, the connector is located proximal to the entry point and in a second position, the connector is located distal to the entry point.

In accordance with another embodiment, a device support for providing support to an elongated medical device between a first device module and a second device module coupled to a linear member of a robotic drive of a catheter-based procedure system includes a first tube having a lengthwise slit configured to move between a first position and a second position and a second tube having a lengthwise opening, an inner diameter and an outer diameter. The first tube has an inner diameter and an outer diameter. The second tube is disposed around the outer diameter of the first tube and is configured to provide a force on the first tube to hold the first tube in the first position.

In accordance with another embodiment, a cassette for use in a robotic drive of a catheter-based procedure system includes a housing having a distal end and a proximal end, an entry point to a device support on the distal end of the housing; and a modular section of the housing located between the proximal end and the entry point on the distal end. The modular section is configured to receive a plurality of different adapters configured to support different elongated medical devices.

In accordance with another embodiment, an apparatus for providing support for an elongated medical device in a catheter-based procedure system, the apparatus includes a cassette and an elongated medical device adapter. The cassette includes a housing having a distal end and a proximal end, an entry point to a device support on the distal end of the housing and a modular section of the housing located between the proximal end and the entry point on the distal end. The modular section includes a midsection and a recess positioned off-axis from a longitudinal axis of the cassette. The elongated medical device adapter includes a first section configured to receive a first elongated medical device and a second section configured to receive a second elongated medical device. The second section is positioned at an angle from a longitudinal axis of the first section. The first section of the elongated medical device adapter is positioned in the midsection of the modular section and the second section of the elongated medical device adapter is positioned in the recess of the modular section.

In accordance with another embodiment, a cassette for use in a robotic drive of a catheter-based procedure system includes a rigid support including an opening and an isolated interface positioned within the opening. The isolated interface includes a cradle for an elongated medical device. The recess and the isolated interface may allow a limited range of motion of the isolated interface in the x, y, and z directions relative to the rigid support.

In accordance with another embodiment, a cassette for use in a robotic drive of a catheter-based procedure system includes a rigid support portion, an interface portion configured to support a hemostasis valve having a port and an apparatus for anchoring a fluid connection to the hemostasis valve. The apparatus for anchoring a fluid connection includes a flexible tube having a first end and a second end a clip attached to the rigid support portion and the second end of the flexible tube. The first end of the flexible tube is configured to connect to the port of the hemostasis valve.

### BRIEF DESCRIPTION OF THE DRAWINGS

The invention will become more fully understood from the following detailed description, taken in conjunction with the accompanying drawings, wherein the reference numerals refer to like parts in which:

FIG. 1 is a perspective view of an exemplary catheter procedure system in accordance with an embodiment;

FIG. 2 is a schematic block diagram of an exemplary catheter procedure system in accordance with an embodiment;

FIG. 3 is a perspective view of a drive assembly for a catheter procedure system in accordance with an embodiment;

FIG. 4 is a perspective view of device supports with fixed front (or distal) and rear (or proximal) points to provide tension in accordance with an embodiment;

FIG. 5 is a diagram showing a top view of a cassette with a device support in a withdrawn position to facilitate exchange of an elongated medical device in accordance with an embodiment;

FIG. 6 is a diagram showing a top view of a cassette with a device support in an extended position constrained at two ends in accordance with an embodiment;

FIG. 7 is a top view of two device modules with device supports in accordance with an embodiment;

FIG. 8 is a top view illustrating forward translation of a device module linearly relative to a device support in accordance with an embodiment;

FIG. 9 is a top view illustrating reverse translation of a device module linearly relative to a device support in accordance with an embodiment;

FIG. 10 is a top view illustrating reverse translation of a device module linearly relative to a device support in accordance with an embodiment;

FIG. 11 shows a simplified top view of four device modules and four device supports for a robotic drive in accordance with an embodiment;

FIG. 12 shows a simplified top view illustrating movement of a device module relative to a device support in accordance with an embodiment;

FIG. 13 shows a simplified top view illustrating the four device modules of FIG. 11 in a forward position relative to their respective device support in accordance with an embodiment;

FIG. 14 shows a simplified top view illustrating the four device modules of FIG. 11 in a withdrawn position relative to their respective device support in accordance with an embodiment;

FIG. 15 is a side view of a proximal end of a device support that is extended and a rear constraint for a rear (or proximal) fixed point to which the device support is connected in accordance with an embodiment;

FIG. 16 is a side view of a proximal end of a device support that is partially retracted and a rear constraint for a rear (or proximal) fixed point to which the device support is connected in accordance with an embodiment;

FIG. 17 shows a simplified top view of device modules with device supports stored on a reel in accordance with an embodiment;

FIG. 18 shows an exemplary spooled tensioner in accordance with an embodiment;

FIG. 19 shows a simplified top view of device modules with drive device supports in accordance with an embodiment;

FIG. 20 shows an exemplary geared tensioner in accordance with an embodiment;

FIG. 21 shows a simplified top view of device modules with device supports formed with accordions or springs in accordance with an embodiment;

FIG. 22 illustrates a compressed accordion/spring in accordance with an embodiment;

FIG. 23 illustrates a stretched accordion/spring in accordance with an embodiment;

FIGS. 24 (a)-(c) are perspective views of exemplary slit shapes for a device support flexible tube in accordance with an embodiment;

FIG. 25 is an exploded view of a device module and an elongated medical device in accordance with an embodiment;

FIG. 26a is a perspective view of a cassette with a device support installed and in a retracted position in accordance with an embodiment;

FIG. 26b is a perspective view of a cassette with a device support installed and in a retracted position in accordance with an embodiment;

FIG. 27 is a top view of a device support and connector extended from a cassette ahead of an EMD entry point in accordance with an embodiment;

FIG. 28 is a top view of a device support and connector withdrawn behind an EMD entry point in accordance with an embodiment;

FIG. 29 is an end view of a splitter holding open a device support in accordance with an embodiment;

FIG. 30 is a top view of cassette with a device support connector withdrawn and off of a device axis to facilitate loading of an EMD in accordance with an embodiment;

FIG. 31 is a perspective view of a forward constraint and a connector in accordance with an embodiment;

FIG. 32 is a perspective view of a forward constraint with a lid in accordance with an embodiment;

FIG. 33 is a perspective view of a distal support arm and distal support connection in accordance with an embodiment;

FIG. 34 is a perspective view of a distal support connection coupled to a device support and connector in accordance with an embodiment;

FIG. 35 is a side view of a distal support arm, distal support connection and an introducer interface support in accordance with an embodiment;

FIG. 36 is a perspective view of an introducer interface support connected to an introducer sheath in accordance with an embodiment;

FIG. 37 is a perspective view of a movable distal support arm in a first position in accordance with an embodiment;

FIG. 38 is a perspective view of a moveable distal support arm in a second position in accordance with an embodiment;

FIG. 39 is a top view of a moveable distal support arm and movable support arm in a first position in accordance with an embodiment;

FIG. 40 is a top view of a moveable distal support arm and movable support arm in a second position in accordance with an embodiment;

FIG. 41 is a top view illustrating movement of a distal support arm and a support arm from the second position to the first position in accordance with an embodiment;

FIG. 42 is a perspective view of a catheter with an on-device adapter in accordance with an embodiment;

FIG. 43 is a perspective view of a guidewire with an on-device adapter in accordance with an embodiment;

FIG. 44 is a perspective view of a cassette with an installed elongated medical device with an on-device adapter in accordance with an embodiment;

FIG. 45 is a exploded view of a cassette and an elongated medical device with an on-device adapter that is removed from the cassette in accordance with an embodiment;

FIG. 46 s a top view of a cassette in accordance with an embodiment;

FIG. 47 is an exploded view of an elongated medical device (EMD) adapter and a lid in accordance with an embodiment;

FIG. 48 is a perspective view of an EMD adapter and EMD installed in a cassette in accordance with an embodiment;

FIG. 49 is a top view of s cassette with a floating interface and a rigid support section in accordance with an embodiment;

FIG. 50a is an end cross-sectional view of an floating (or isolated) interface and rigid support section of a cassette in accordance with an embodiment;

FIG. 50b is an exploded isometric view of a cassette showing a first component and a second component of a floating (or isolated) interface in accordance with an embodiment;

FIG. 51 is a bottom view of the floating (or isolated) interface of a cassette in accordance with an embodiment;

FIG. 52 shows cradle supporting a rotational drive gear with rollers in accordance with an embodiment; and

FIG. 53 illustrates a cassette with a support assembly for anchoring tubing and fluid connections in accordance with an embodiment;

FIG. 54 is an end cross-sectional view of a device support in accordance with an embodiment; and

FIG. 55 is an end cross-sectional vie of a device support and splitter in accordance with an embodiment.

### DETAILED DESCRIPTION

The following definitions will be used herein. The term elongated medical device (EMD) refers to, but is not limited to, catheters (e.g. guide catheters, microcatheters, balloon/stent catheters), wire-based devices (guidewires, embolization coils, stent retrievers, etc.), and devices that have a combination of these. Wire-based EMD includes, but is not limited to, guidewires, microwires, a proximal pusher for embolization coils, stent retrievers, self-expanding stents, and flow divertors. Typically wire-based EMD's do not have a hub or handle at its proximal terminal end. In one embodiment the EMD is a catheter having a hub at a proximal end of the catheter and a flexible shaft extending from the hub toward the distal end of the catheter, wherein the shaft is more flexible than the hub. In one embodiment the catheter includes an intermediary portion that transitions between the hub and the shaft that has an intermediate flexibility that is less rigid than the hub and more rigid than the shaft. In one embodiment the intermediary portion is a strain relief.

The terms distal and proximal define relative locations of two different features. With respect to a robotic drive the terms distal and proximal are defined by the position of the robotic drive in its intended use relative to a patient. When used to define a relative position, the distal feature is the feature of the robotic drive that is closer to the patient than a proximal feature when the robotic drive is in its intended in-use position. Within a patient, any vasculature landmark further away along the path from the access point is considered more distal than a landmark closer to the access point, where the access point is the point at which the EMD enters the patient. Similarly, the proximal feature is the feature that is farther from the patient than the distal feature when the robotic drive in its intended in-use position. When used to define direction, the distal direction refers to a path on which something is moving or is aimed to move or along which something is pointing or facing from a proximal feature toward a distal feature and/or patient when the robotic drive is in its intended in-use position. The proximal direction is the opposite direction of the distal direction.

The term longitudinal axis of a member (e.g., an EMD or other element in the catheter-based procedure system) is the direction of orientation going from a proximal portion of the member to a distal portion of the member. By way of example, the longitudinal axis of a guidewire is the direction of orientation from a proximal portion of the guide wire toward a distal portion of the guidewire even though the guidewire may be non-linear in the relevant portion. The term axial movement of a member refers to translation of the member along the longitudinal axis of the member. When a distal end of an EMD is axially moved in a distal direction along its longitudinal axis into or further into the patient, the EMD is being advanced. When the distal end of an EMD is axially moved in a proximal direction along its longitudinal axis out of or further out of the patient, the EMD is being withdrawn. The term rotational movement of a member refers to change in angular orientation of the member about the local longitudinal axis of the member. Rotational movement of an EMD corresponds to clockwise or counterclockwise rotation of the EMD about its longitudinal axis due to an applied torque.

The term axial insertion refers to inserting a first member into a second member along the longitudinal axes of the second member. The term lateral insertion refers to inserting a first member into a second member along a direction in a plane perpendicular to the longitudinal axis of the second member. This can also be referred to as radial loading or side loading. The term pinch refers to releasably fixing an EMD to a member such that the EMD and member move together when the member moves. The term unpinch refers to releasing the EMD from a member such that the EMD and member move independently when the member moves. The term clamp refers to releasably fixing an EMD to a member such that the EMD's movement is constrained with respect to the member. The member can be fixed with respect to a global coordinate system or with respect to a local coordinate system. The term unclamp refers to releasing the EMD from the member such that the EMD can move independently.

The term grip refers to the application of a force or torque to an EMD by a drive mechanism that causes motion of the EMD without slip in at least one degree of freedom. The term ungrip refers to the release of the application of force or torque to the EMD by a drive mechanism such that the position of the EMD is no longer constrained. In one example, an EMD gripped between two tires will rotate about its longitudinal axis when the tires move longitudinally relative to one another. The rotational movement of the EMD is different than the movement of the two tires. The position of an EMD that is gripped is constrained by the drive mechanism. The term buckling refers to the tendency of a flexible EMD when under axial compression to bend away from the longitudinal axis or intended path along which it is being advanced. In one embodiment axial compression occurs in response to resistance from being navigated in the vasculature. The distance an EMD may be driven along its longitudinal axis without support before the EMD buckles is referred to herein as the device buckling distance. The device buckling distance is a function of the device's stiffness, geometry (including but not limited to diameter), and force being applied to the EMD. Buckling may cause the EMD to form an arcuate portion different than the intended path. Kinking is a case of buckling in which deformation of the EMD is non-elastic resulting in a permanent set.

The terms top, up, and upper refer to the general direction away from the direction of gravity and the terms bottom, down, and lower refer to the general direction in the direction of gravity. The term inwardly refers to the inner portion of a feature. The term outwardly refers to the outer portion of a feature. The term sterile interface refers to an interface or boundary between a sterile and non-sterile unit. For example, a cassette may be a sterile interface between the robotic drive and at least one EMD. The term sterilizable unit refers to an apparatus that is capable of being sterilized (free from pathogenic microorganisms). This includes, but is not limited to, a cassette, consumable unit, drape, device adapter, and sterilizable drive modules/units (which may include electromechanical components). Sterilizable Units may come into contact with the patient, other sterile devices, or anything else placed within the sterile field of a medical procedure.

The term on-device adapter refers to sterile apparatus capable of releasably pinching an MED to provide a driving interface. For example, the on-device adapter is also known as an end-effector or EMD capturing device. In one non-limiting embodiment, the on-device adapter is a collet that is operatively controlled robotically to rotate the EMD about its longitudinal axis, to pinch and/or unpinch the EMD to the collet, and/or to translate the EMD along its longitudinal axis. In one embodiment the on-device adapter is a hub-drive mechanism such as a driven gear located on the hub of an EMD. The term hub driving or proximal driving refers to holding on to and manipulating an EMD from a proximal position (e.g., geared adapter on catheter hub). In one embodiment, hub driving refers to imparting a force or torque to the hub of a catheter to translate and/or rotate the catheter. In hub driving, often applying typical clinical loads causes the EMD to buckle and thus hub driving often requires anti-buckling features in the driving mechanism. For devices that do not have hubs or other interfaces (e.g., a guidewire), device adapters may be added to the device to act as a temporary hub. In one embodiment, an EMD handle includes mechanisms to manipulate features within the catheter such as wires that extend from the handle to the distal end of the catheter to deflect the distal end of the catheter. In contrast, the hub is the rigid portion of the EMD at the proximal end that does not include control mechanisms to manipulate features within the catheter. The term shaft (distal) driving refers to holding on to and manipulating an EMD along its shaft. For example, an on-device adapter may be placed just proximal of the hub or Y-connector the device is inserted into. If the location of the on-device adapter is at the proximity of an insertion point (to the body or another catheter or valve), shaft driving does not typically require anti-buckling features (it may include anti-buckling features to improve drive capability).

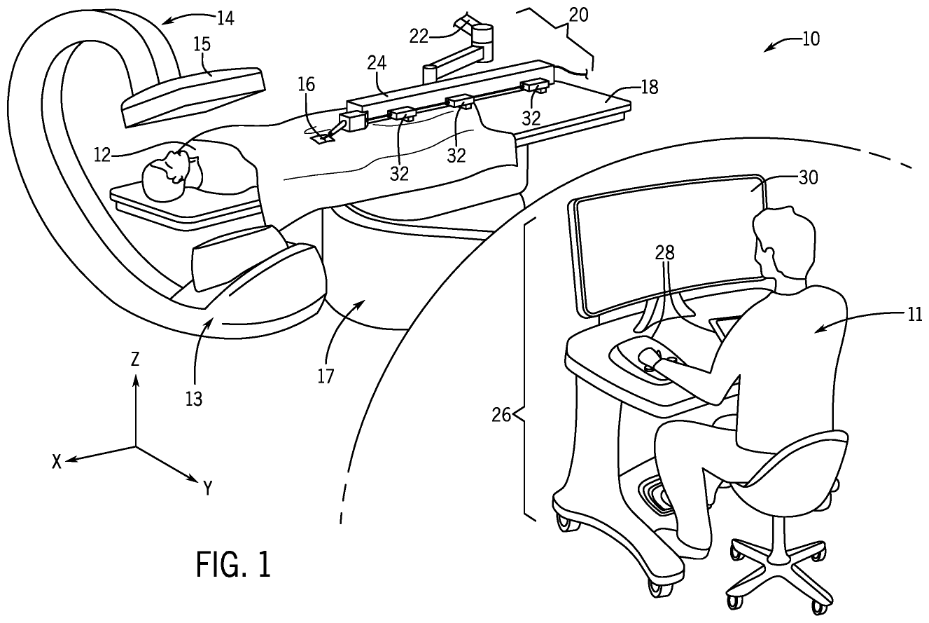

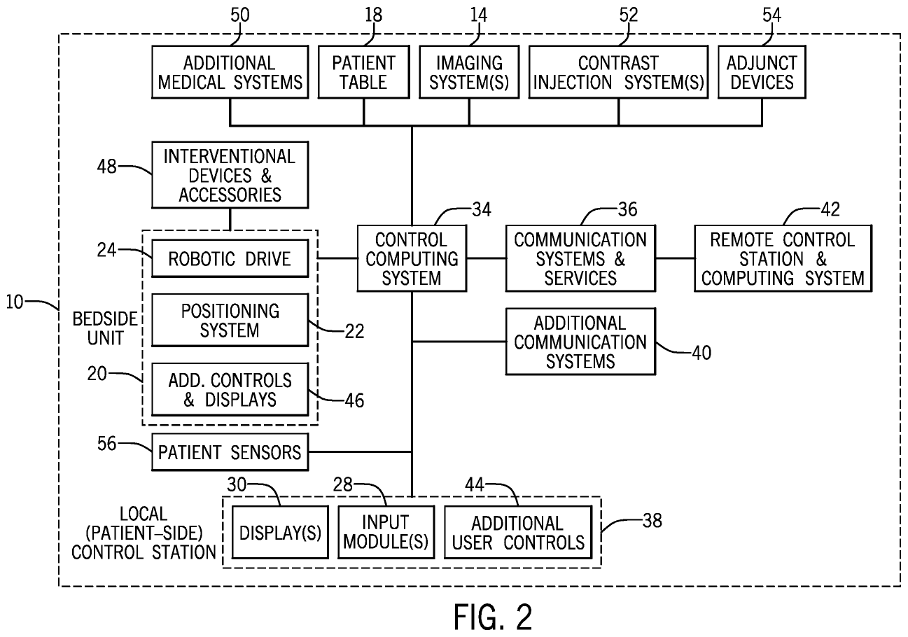

FIG. 1 is a perspective view of an exemplary catheter-based procedure system 10 in accordance with an embodiment. Catheter-based procedure system 10 may be used to perform catheter-based medical procedures, e.g., percutaneous intervention procedures such as a percutaneous coronary intervention (PCI) (e.g., to treat STEMI), a neurovascular interventional procedure (NVI) (e.g., to treat an emergent large vessel occlusion (ELVO)), peripheral vascular intervention procedures (PVI) (e.g., for critical limb ischemia (CLI), etc.). Catheter-based medical procedures may include diagnostic catheterization procedures during which one or more catheters or other elongated medical devices (EMDs) are used to aid in the diagnosis of a patient's disease. For example, during one embodiment of a catheter-based diagnostic procedure, a contrast media is injected onto one or more arteries through a catheter and an image of the patient's vasculature is taken. Catheter-based medical procedures may also include catheter-based therapeutic procedures (e.g., angioplasty, stent placement, treatment of peripheral vascular disease, clot removal, arterial venous malformation therapy, treatment of aneurysm, etc.) during which a catheter (or other EMD) is used to treat a disease. Therapeutic procedures may be enhanced by the inclusion of adjunct devices 54 (shown in FIG. 2) such as, for example, intravascular ultrasound (IVUS), optical coherence tomography (OCT), fractional flow reserve (FFR), etc. It should be noted, however, that one skilled in the art would recognize that certain specific percutaneous intervention devices or components (e.g., type of guidewire, type of catheter, etc.) may be selected based on the type of procedure that is to be performed. Catheter-based procedure system 10 can perform any number of catheter-based medical procedures with minor adjustments to accommodate the specific percutaneous intervention devices to be used in the procedure.

Catheter-based procedure system 10 includes, among other elements, a bedside unit 20 and a control station 26. Bedside unit 20 includes a robotic drive 24 and a positioning system 22 that are located adjacent to a patient 12. Patient 12 is supported on a patient table 18. The positioning system 22 is used to position and support the robotic drive 24. The positioning system 22 may be, for example, a robotic arm, an articulated arm, a holder, etc. The positioning system 22 may be attached at one end to, for example, a rail on the patient table 18, a base, or a cart. The other end of the positioning system 22 is attached to the robotic drive 24. The positioning system 22 may be moved out of the way (along with the robotic drive 24) to allow for the patient 12 to be placed on the patient table 18. Once the patient 12 is positioned on the patient table 18, the positioning system 22 may be used to situate or position the robotic drive 24 relative to the patient 12 for the procedure. In an embodiment, patient table 18 is operably supported by a pedestal 17, which is secured to the floor and/or earth. Patient table 18 is able to move with multiple degrees of freedom, for example, roll, pitch, and yaw, relative to the pedestal 17. Bedside unit 20 may also include controls and displays 46 (shown in FIG. 2). For example, controls and displays may be located on a housing of the robotic drive 24.

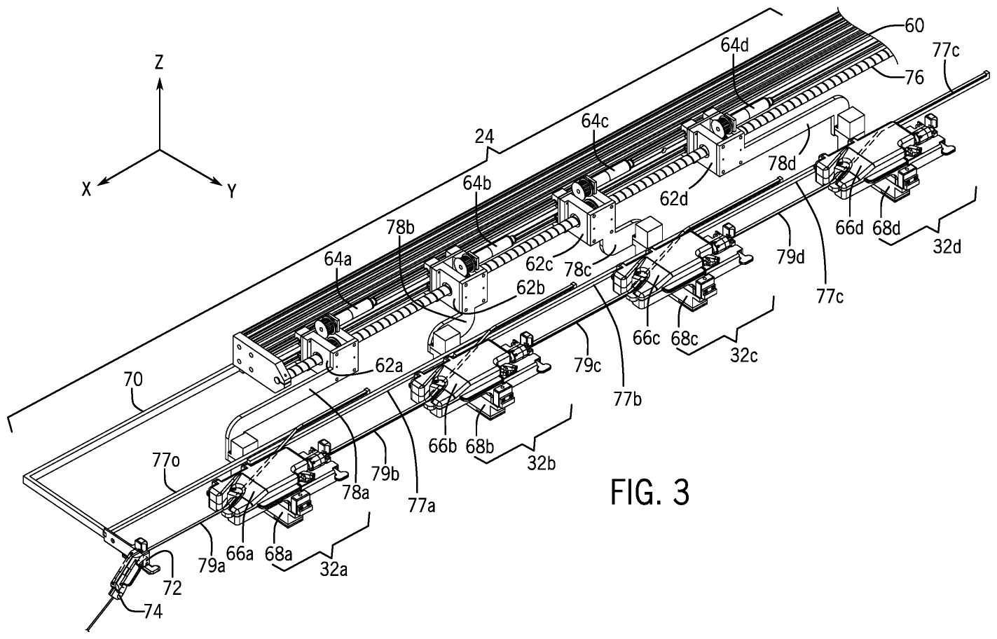

Generally, the robotic drive 24 may be equipped with the appropriate percutaneous interventional devices and accessories 48 (shown in FIG. 2) (e.g., guidewires, various types of catheters including balloon catheters, stent delivery systems, stent retrievers, embolization coils, liquid embolics, aspiration pumps, device to deliver contrast media, medicine, hemostasis valve adapters, syringes, stopcocks, inflation device, etc.) to allow the user or operator 11 to perform a catheter-based medical procedure via a robotic system by operating various controls such as the controls and inputs located at the control station 26. Bedside unit 20, and in particular robotic drive 24, may include any number and/or combination of components to provide bedside unit 20 with the functionality described herein. A user or operator 11 at control station 26 is referred to as the control station user or control station operator and referred to herein as user or operator. A user or operator at bedside unit 20 is referred to as bedside unit user or bedside unit operator. The robotic drive 24 includes a plurality of device modules 32a-d mounted to a rail or linear member 60 (shown in FIG. 3). The rail or linear member 60 guides and supports the device modules. Each of the device modules 32a-d may be used to drive an EMD such as a catheter or guidewire. For example, the robotic drive 24 may be used to automatically feed a guidewire into a diagnostic catheter and into a guide catheter in an artery of the patient 12. One or more devices, such as an EMD, enter the body (e.g., a vessel) of the patient 12 at an insertion point 16 via, for example, an introducer sheath.

Bedside unit 20 is in communication with control station 26, allowing signals generated by the user inputs of control station 26 to be transmitted wirelessly or via hardwire to bedside unit 20 to control various functions of bedside unit 20. As discussed below, control station 26 may include a control computing system 34 (shown in FIG. 2) or be coupled to the bedside unit 20 through a control computing system 34. Bedside unit 20 may also provide feedback signals (e.g., loads, speeds, operating conditions, warning signals, error codes, etc.) to control station 26, control computing system 34 (shown in FIG. 2), or both. Communication between the control computing system 34 and various components of the catheter-based procedure system 10 may be provided via a communication link that may be a wireless connection, cable connections, or any other means capable of allowing communication to occur between components. Control station 26 or other similar control system may be located either at a local site (e.g., local control station 38 shown in FIG. 2) or at a remote site (e.g., remote control station and computer system 42 shown in FIG. 2). Catheter procedure system 10 may be operated by a control station at the local site, a control station at a remote site, or both the local control station and the remote control station at the same time. At a local site, user or operator 11 and control station 26 are located in the same room or an adjacent room to the patient 12 and bedside unit 20. As used herein, a local site is the location of the bedside unit 20 and a patient 12 or subject (e.g., animal or cadaver) and the remote site is the location of a user or operator 11 and a control station 26 used to control the bedside unit 20 remotely. A control station 26 (and a control computing system) at a remote site and the bedside unit 20 and/or a control computing system at a local site may be in communication using communication systems and services 36 (shown in FIG. 2), for example, through the Internet. In an embodiment, the remote site and the local (patient) site are away from one another, for example, in different rooms in the same building, different buildings in the same city, different cities, or other different locations where the remote site does not have physical access to the bedside unit 20 and/or patient 12 at the local site.

Control station 26 generally includes one or more input modules 28 configured to receive user inputs to operate various components or systems of catheter-based procedure system 10. In the embodiment shown, control station 26 allows the user or operator 11 to control bedside unit 20 to perform a catheter-based medical procedure. For example, input modules 28 may be configured to cause bedside unit 20 to perform various tasks using percutaneous intervention devices (e.g., EMDs) interfaced with the robotic drive 24 (e.g., to advance, retract, or rotate a guidewire, advance, retract or rotate a catheter, inflate or deflate a balloon located on a catheter, position and/or deploy a stent, position and/or deploy a stent retriever, position and/or deploy a coil, inject contrast media into a catheter, inject liquid embolics into a catheter, inject medicine or saline into a catheter, aspirate on a catheter, or to perform any other function that may be performed as part of a catheter-based medical procedure). Robotic drive 24 includes various drive mechanisms to cause movement (e.g., axial and rotational movement) of the components of the bedside unit 20 including the percutaneous intervention devices.

In one embodiment, input modules 28 may include one or more touch screens, joysticks, scroll wheels, and/or buttons. In addition to input modules 28, the control station 26 may use additional user controls 44 (shown in FIG. 2) such as foot switches and microphones for voice commands, etc. Input modules 28 may be configured to advance, retract, or rotate various components and percutaneous intervention devices such as, for example, a guidewire, and one or more catheters or microcatheters. Buttons may include, for example, an emergency stop button, a multiplier button, device selection buttons and automated move buttons. When an emergency stop button is pushed, the power (e.g., electrical power) is shut off or removed to bedside unit 20. When in a speed control mode, a multiplier button acts to increase or decrease the speed at which the associated component is moved in response to a manipulation of input modules 28. When in a position control mode, a multiplier button changes the mapping between input distance and the output commanded distance. Device selection buttons allow the user or operator 11 to select which of the percutaneous intervention devices loaded into the robotic drive 24 are controlled by input modules 28. Automated move buttons are used to enable algorithmic movements that the catheter-based procedure system 10 may perform on a percutaneous intervention device without direct command from the user or operator 11. In one embodiment, input modules 28 may include one or more controls or icons (not shown) displayed on a touch screen (that may or may not be part of a display 30), that, when activated, causes operation of a component of the catheter-based procedure system 10. Input modules 28 may also include a balloon or stent control that is configured to inflate or deflate a balloon and/or deploy a stent. Each of the input modules 28 may include one or more buttons, scroll wheels, joysticks, touch screen, etc. that may be used to control the particular component or components to which the control is dedicated. In addition, one or more touch screens may display one or more icons (not shown) related to various portions of input modules 28 or to various components of catheter-based procedure system 10.

Control station 26 may include a display 30. In other embodiments, the control station 26 may include two or more displays 30. Display 30 may be configured to display information or patient specific data to the user or operator 11 located at control station 26. For example, display 30 may be configured to display image data (e.g., X-ray images, MRI images, CT images, ultrasound images, etc.), hemodynamic data (e.g., blood pressure, heart rate, etc.), patient record information (e.g., medical history, age, weight, etc.), lesion or treatment assessment data (e.g., IVUS, OCT, FFR, etc.). In addition, display 30 may be configured to display procedure specific information (e.g., procedural checklist, recommendations, duration of procedure, catheter or guidewire position, volume of medicine or contrast agent delivered, etc.). Further, display 30 may be configured to display information to provide the functionalities associated with control computing system 34 (shown in FIG. 2). Display 30 may include touch screen capabilities to provide some of the user input capabilities of the system.

Catheter-based procedure system 10 also includes an imaging system 14. Imaging system 14 may be any medical imaging system that may be used in conjunction with a catheter based medical procedure (e.g., non-digital X-ray, digital X-ray, CT, MRI, ultrasound, etc.). In an exemplary embodiment, imaging system 14 is a digital X-ray imaging device that is in communication with control station 26. In one embodiment, imaging system 14 may include a C-arm (shown in FIG. 1) that allows imaging system 14 to partially or completely rotate around patient 12 in order to obtain images at different angular positions relative to patient 12 (e.g., sagittal views, caudal views, anterior-posterior views, etc.). In one embodiment imaging system 14 is a fluoroscopy system including a C-arm having an X-ray source 13 and a detector 15, also known as an image intensifier.

Imaging system 14 may be configured to take X-ray images of the appropriate area of patient 12 during a procedure. For example, imaging system 14 may be configured to take one or more X-ray images of the head to diagnose a neurovascular condition. Imaging system 14 may also be configured to take one or more X-ray images (e.g., real time images) during a catheter-based medical procedure to assist the user or operator 11 of control station 26 to properly position a guidewire, guide catheter, microcatheter, stent retriever, coil, stent, balloon, etc. during the procedure. The image or images may be displayed on display 30. For example, images may be displayed on display 30 to allow the user or operator 11 to accurately move a guide catheter or guidewire into the proper position.

In order to clarify directions, a rectangular coordinate system is introduced with X, Y, and Z axes. The positive X axis is oriented in a longitudinal (axial) distal direction, that is, in the direction from the proximal end to the distal end, stated another way from the proximal to distal direction. The Y and Z axes are in a transverse plane to the X axis, with the positive Z axis oriented up, that is, in the direction opposite of gravity, and the Y axis is automatically determined by right-hand rule.

FIG. 2 is a block diagram of catheter-based procedure system 10 in accordance with an exemplary embodiment. Catheter-procedure system 10 may include a control computing system 34. Control computing system 34 may physically be, for example, part of control station 26 (shown in FIG. 1). Control computing system 34 may generally be an electronic control unit suitable to provide catheter-based procedure system 10 with the various functionalities described herein. For example, control computing system 34 may be an embedded system, a dedicated circuit, a general-purpose system programmed with the functionality described herein, etc. Control computing system 34 is in communication with bedside unit 20, communications systems and services 36 (e.g., Internet, firewalls, cloud services, session managers, a hospital network, etc.), a local control station 38, additional communications systems 40 (e.g., a telepresence system), a remote control station and computing system 42, and patient sensors 56 (e.g., electrocardiogram (ECG) devices, electroencephalogram (EEG) devices, blood pressure monitors, temperature monitors, heart rate monitors, respiratory monitors, etc.). The control computing system is also in communication with imaging system 14, patient table 18, additional medical systems 50, contrast injection systems 52 and adjunct devices 54 (e.g., IVUS, OCT, FFR, etc.). The bedside unit 20 includes a robotic drive 24, a positioning system 22 and may include additional controls and displays 46. As mentioned above, the additional controls and displays may be located on a housing of the robotic drive 24. Interventional devices and accessories 48 (e.g., guidewires, catheters, etc.) interface to the bedside system 20. In an embodiment, interventional devices and accessories 48 may include specialized devices (e.g., IVUS catheter, OCT catheter, FFR wire, diagnostic catheter for contrast, etc.) which interface to their respective adjunct devices 54, namely, an IVUS system, an OCT system, and FFR system, etc.

In various embodiments, control computing system 34 is configured to generate control signals based on the user's interaction with input modules 28 (e.g., of a control station 26 (shown in FIG. 1) such as a local control station 38 or a remote control station 42) and/or based on information accessible to control computing system 34 such that a medical procedure may be performed using catheter-based procedure system 10. The local control station 38 includes one or more displays 30, one or more input modules 28, and additional user controls 44. The remote control station and computing system 42 may include similar components to the local control station 38. The remote 42 and local 38 control stations can be different and tailored based on their required functionalities. The additional user controls 44 may include, for example, one or more foot input controls. The foot input control may be configured to allow the user to select functions of the imaging system 14 such as turning on and off the X-ray and scrolling through different stored images. In another embodiment, a foot input device may be configured to allow the user to select which devices are mapped to scroll wheels included in input modules 28. Additional communication systems 40 (e.g., audio conference, video conference, telepresence, etc.) may be employed to help the operator interact with the patient, medical staff (e.g., angio-suite staff), and/or equipment in the vicinity of the bedside.

Catheter-based procedure system 10 may be connected or configured to include any other systems and/or devices not explicitly shown. For example, catheter-based procedure system 10 may include image processing engines, data storage and archive systems, automatic balloon and/or stent inflation systems, medicine injection systems, medicine tracking and/or logging systems, user logs, encryption systems, systems to restrict access or use of catheter-based procedure system 10, etc.

As mentioned, control computing system 34 is in communication with bedside unit 20 which includes a robotic drive 24, a positioning system 22 and may include additional controls and displays 46, and may provide control signals to the bedside unit 20 to control the operation of the motors and drive mechanisms used to drive the percutaneous intervention devices (e.g., guidewire, catheter, etc.). The various drive mechanisms may be provided as part of a robotic drive 24. FIG. 3 is a perspective view of a robotic drive for a catheter-based procedure system 10 in accordance with an embodiment. In FIG. 3, a robotic drive 24 includes multiple device modules 32a-d coupled to a linear member 60. Each device module 32a-d is coupled to the linear member 60 via a stage 62a-d moveably mounted to the linear member 60. A device module 32a-d may be connected to a stage 62a-d using a connector such as an offset bracket 78a-d. In another embodiment, the device module 32a-d is directly mounted to the stage 62a-d. Each stage 62a-d may be independently actuated to move linearly along the linear member 60. Accordingly, each stage 62a-d (and the corresponding device module 32a-d coupled to the stage 62a-d) may independently move relative to each other and the linear member 60. A drive mechanism is used to actuate each stage 62a-d. In the embodiment shown in FIG. 3, the drive mechanism includes independent stage translation motors 64a-d coupled to each stage 62a-d and a stage drive mechanism 76, for example, a lead screw via a rotating nut, a rack via a pinion, a belt via a pinion or pulley, a chain via a sprocket, or the stage translation motors 64a-d may be linear motors themselves. In some embodiments, the stage drive mechanism 76 may be a combination of these mechanisms, for example, each stage 62a-d could employ a different type of stage drive mechanism. In an embodiment where the stage drive mechanism is a lead screw and rotating nut, the lead screw may be rotated and each stage 62a-d may engage and disengage from the lead screw to move, e.g., to advance or retract. In the embodiment shown in FIG. 3, the stages 62a-d and device modules 32a-d are in a serial drive configuration.

Each device module 32a-d includes a drive module 68a-d and a cassette 66a-d mounted on and coupled to the drive module 68a-d. In the embodiment shown in FIG. 3, each cassette 66a-d is mounted to the drive module 68a-d in a vertical orientation. In other embodiments, each cassette 66a-d may be mounted to the drive module 68a-d in other mounting orientations. Each cassette 66a-d is configured to interface with and support a proximal portion of an EMD (not shown). In addition, each cassette 66a-d may include elements to provide one or more degrees of freedom in addition to the linear motion provided by the actuation of the corresponding stage 62a-d to move linearly along the linear member 60. For example, the cassette 66a-d may include elements that may be used to rotate the EMD when the cassette is coupled to the drive module 68a-d. Each drive module 68a-d includes at least one coupler to provide a drive interface to the mechanisms in each cassette 66a-d to provide the additional degree of freedom. Each cassette 66a-d also includes a channel in which a device support 79a-d is positioned, and each device support 79a-d is used to prevent an EMD from buckling. A support arm 77a, 77b, and 77c is attached to each device module 32a, 32b, and 32c, respectively, to provide a fixed point for support of a proximal end of the device supports 79b, 79c, and 79d, respectively. The robotic drive 24 may also include a device support connection 72 connected to a device support 79, a distal support arm 70 and a support arm 77o. Support arm 77o is used to provide a fixed point for support of the proximal end of the distal most device support 79a housed in the distal most device module 32a. In addition, an introducer interface support (redirector) 74 may be connected to the device support connection 72 and an EMD (e.g., an introducer sheath). The configuration of robotic drive 24 has the benefit of reducing volume and weight of the drive robotic drive 24 by using actuators on a single linear member.

To prevent contaminating the patient with pathogens, healthcare staff use aseptic technique in a room housing the bedside unit 20 and the patient 12 or subject (shown in FIG. 1). A room housing the bedside unit 20 and patient 12 may be, for example, a cath lab or an angio suite. Aseptic technique consists of using sterile barriers, sterile equipment, proper patient preparation, environmental controls and contact guidelines. Accordingly, all EMDs and interventional accessories are sterilized and can only be in contact with either sterile barriers or sterile equipment. In an embodiment, a sterile drape (not shown) is placed over the non-sterile robotic drive 24. Each cassette 66a-d is sterilized and acts as a sterile interface between the draped robotic drive 24 and at least one EMD. Each cassette 66a-d can be designed to be sterile for single use or to be re-sterilized in whole or part so that the cassette 66a-d or its components can be used in multiple procedures.

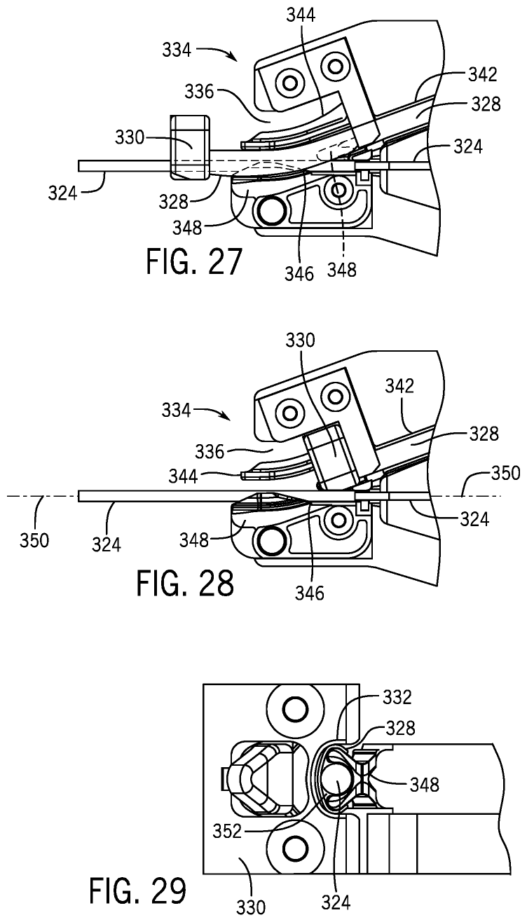

As mentioned, the robotic drive 24 may include a device support 79a-d between each device module 32a-d and between the most distal device module 32a and the device support connection 72. Each device support 79a-d is configured to prevent elongated medical devices from buckling as they are advanced outside of a patient and prior to being advanced into a more distal EMD. In an embodiment, each device support 79a-d may be a flexible tube with a lengthwise slit and used in connection with a splitter on a cassette. Each device support 79a-d is fixed or constrained at both ends so that the device support may be kept in tension so that the flexible tube is limited in the amount of displacement it can buckle. Buckling the elongated medical device limits the amount of force that can be applied and can permanently damage the elongated medical device. The compressive load can be caused by several factors, which may include friction between the EMD and device support, friction between the device support and a cassette (e.g. a splitter in the cassette (discussed below with respect to FIGS. 27-29)), etc. Maintaining the device support under tension may eliminate the need for extra column strength and allow for smaller, more flexible device supports. In one embodiment where the device support is a flexible tube, tension may be provided by fixing or constraining a front (or distal) and rear (or proximal) point or location of the flexible tube. The device supports 79a-d shown in FIG. 3 are one embodiment of a device support with fixed front and rear points. In another embodiment, the device support may be an accordion or spring type support that provides appropriate tension. Each of these different embodiments of a device support are discussed further below.

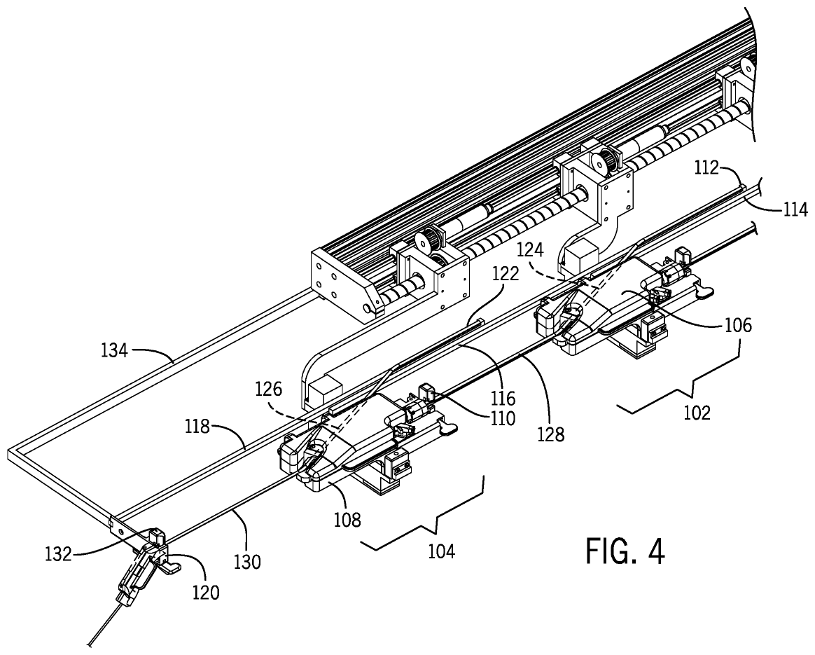

FIG. 4 is a perspective view of device supports with fixed front (or distal) and rear (or proximal) points to provide appropriate tension in accordance with an embodiment. FIG. 4 illustrates the device support embodiment shown in FIG. 3. In FIG. 4, a first device module 102 includes a first cassette 106 that has a first device support 128, e.g., a flexible tube, positioned in a channel 124 of the cassette 106. The first cassette 106 and the first device support 128 are moveable relative to one another. In FIG. 4, the first device support 128 extends out from the distal end of the first cassette 106 and a first end of the first device support 128 connects to a proximal end of a second device module 104 at a first front (or distal) fixed point 110. The second device module 104 is located distal to the first device module 102. The second device module 104 includes a second cassette 108 and a support arm 116 that extends from the second device module 104 in a proximal direction towards the first cassette 106. A second end of the first device support 128 extends out from the proximal end of the first cassette 106 and connects to a first rear (or proximal) fixed point 112 on a proximal end of the support arm 116 of the second device module 104. The first device support 128 is held in place by fixed (or constrained) first front 110 and rear 112 points. The first front and rear fixed points 110 and 112 are kept a constant distance from one another. The first front and rear fixed points 110 and 112 may be rigid or may have some elasticity to account for manufacturing and assembly tolerance. The first device module 102 also includes a support arm 114 that may be used to provide a rear (or proximal) fixed point for a device support for a cassette (not shown) located proximal to the first cassette 106.

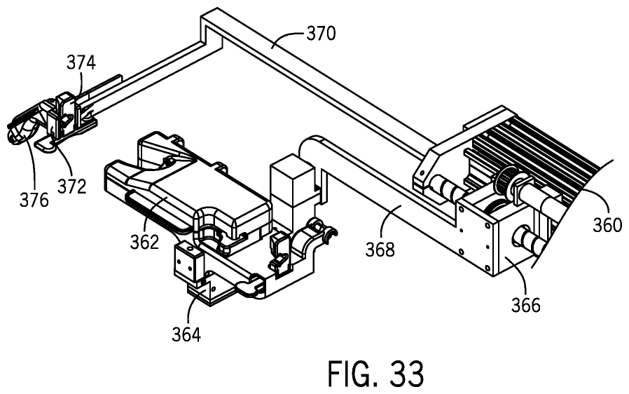

The second device module 104 is the most distal module and closest to the patient (not shown). The second cassette 108 of the second device module 104 includes a second device support 130, e.g., a flexible tube, positioned in a channel 126 of the second cassette 108. The second cassette 108 and the second device support 130 are moveable relative to one another. Since there is no device module or cassette in front of the second device module 104, a distal support connection 132 mounted to a distal support arm 134 is used to provide a second front (or distal) fixed point 120 for the distal end of the second device support 130. The distal support connection 132 and distal support arm 134 are described in further below with regard to FIGS. 33-41. A second end of the second device support 130 extends out from the proximal end of the second cassette 108 and connects to a second rear (or proximal) fixed point 122 on a proximal end of the support arm 118 connected to the distal support arm 134. The second device support 130 is held in tension by fixed second front 120 and rear 122 points. The second front and rear points 120 and 122 are kept a constant distance from one another. The second front and rear fixed points 120 and 122 may be rigid or may have some elasticity to account for manufacturing and assembly tolerance.

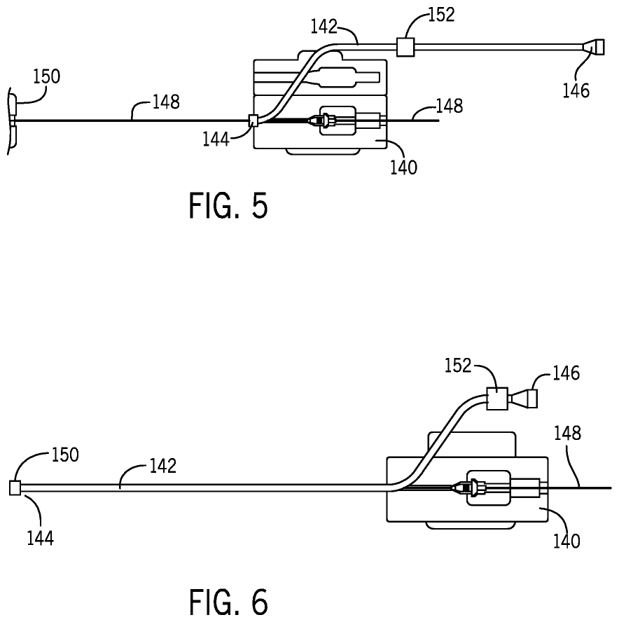

In one embodiment, the distal end of the first device support 128 connected to the first front fixed point 110 and the distal end of the second device support 130 connected to the second front fixed point 120 may be detached or disconnected, as discussed further below, to facilitate loading and unloading of EMDs before, during and after a procedure. FIG. 5 is a diagram showing a top view of a cassette with a device support in a withdrawn position to facilitate exchange of an elongated medical device in accordance with an embodiment. In FIG. 5, a device support 142 of a cassette 140 has been detached from a front (or distal) fixed point 150 and is in a retracted position which exposes an EMD 148 to facilitate loading and unloading of the EMD. As discussed above, the front fixed point 150 is located on a device module distal to the cassette 140. The device support 142 is shown over the cassette 140 cover in FIG. 5 for clarity. A first (or distal) end 144 of the device support 142 is located at the distal end of the cassette 140. A second (or proximal) end 146 of the device support 142 has moved past a rear (or proximal) fixed point 152. As discussed above, the rear fixed point 152 is located on a support arm of, for example, cassette, drive module or stage, distal to the cassette 140. Additionally, the fixed rear point 152 may be attached to the frame of the robotic drive. FIG. 6 is a diagram showing a top view of a cassette with a device support in an extended position constrained at two ends in accordance with an embodiment. When the device support 142 is pulled over the EMD 148, the first end 144 is attached to the front fixed point 150 and the second end 146 is constrained by the rear fixed point 152. As discussed above, the front fixed point 150 and the rear fixed point 152 are fixed relative to a device module the distal end of the EMD 148 is entering. The device support 142 is shown over the cassette 140 cover in FIG. 6 for clarity.

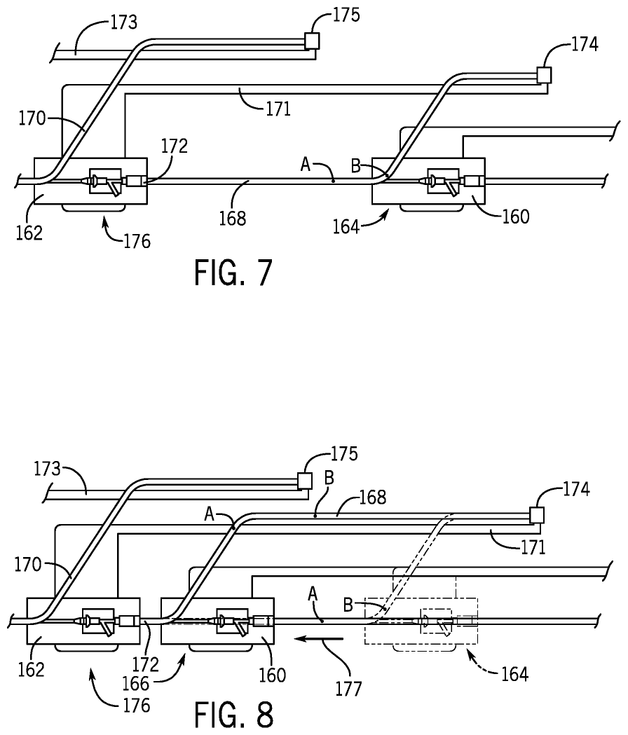

Constraining (fixing) each device support on both ends allows for relative motion between all of the device modules in a robotic drive. FIG. 7 is a top view of two device modules with device supports in accordance with an embodiment. A first device module 160 has a first device support 168 constrained at a first front (or distal) fixed point 172 at the proximal end of a second device module 162 and at a first rear (or proximal) fixed point 174 located on a proximal end of a support arm 171 of the second device module 162. The second device module 162 has a second device support 170 that is constrained at a second front (or distal) fixed point (not shown) and a second rear (or proximal) fixed point 175 located in the proximal end of a support arm 173 of a device module (not shown) distal to the second device module 162. The first device module 160 may be translated forward from a first position 164. The second device module 162 is at a first position 176. FIG. 8 is a top view illustrating forward translation of a device module linearly relative to a device support in accordance with an embodiment. When the first device module 160 moves forward towards the patient (as indicated by arrow 177) from the first position 164 to a second position 166, the first rear fixed point 174 takes the load developed as a cassette of the first device module 160 (and the device module) moves along the first device support 168 (e.g., friction between the cassette and the first device support 168). Accordingly, the first device support 168 will not buckle between the distal end of the cassette on the first device module 160 and the proximal end or rear of a cassette on the second device module 162. As the first device module 160 advances distally toward the second device module 162 (which is stationary at its first position 176 in this example) it moves relative to the first device support 168 as illustrated by reference points A and B located along the length of the first device support 168. When the first device module 160 is at the first position 164, reference point A and reference point B are located proximate to the distal end of the first device module 160. As the first device module 160 advances along the first device support 168, the first device support 168 remains stationary because the second device module 162 to which it is coupled via the first front fixed point 172 and the first rear fixed point 174 is also stationary. When the first device module 160 is located at the second position 166, reference point A and reference point B are located off axis and proximal to the first device module 160. The first device module 160 may also be translated backwards from the second position 166 to the first position 164.

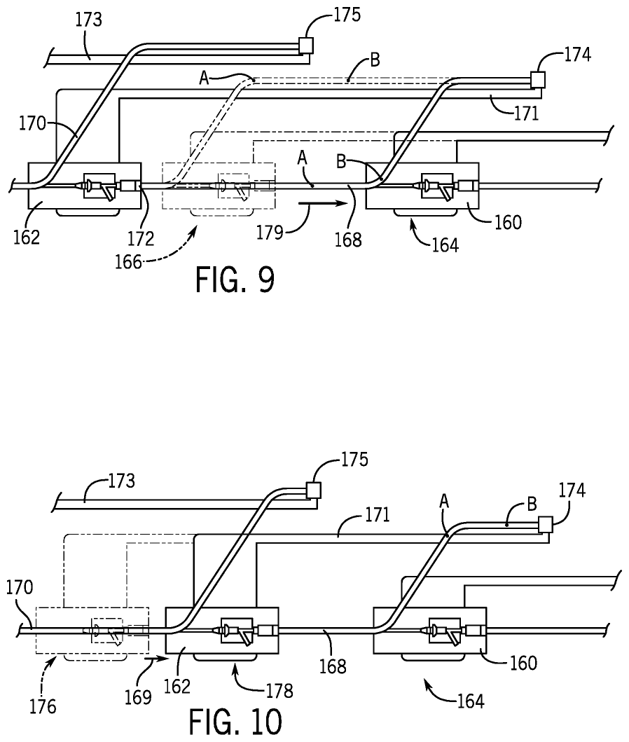

FIG. 9 is a top view illustrating reverse translation of a device module linearly relative to a device support in accordance with an embodiment. When the first device module 160 moves backwards (retracts) away from the patient (as indicated by arrow 179) from the second position 166 to the first position 164, the first front fixed point 172 takes the load developed as a cassette of the first device module 160 (and the device module) moves along the first device support 168 (e.g., friction between the cassette and the first device support 168). Accordingly, the first device support 168 will not buckle between the cassette on the first device module 160 and the first rear fixed point 174. As the first device module 160 moves proximally away from the second device module 162 (which is stationary at its first position 176 in this example) it moves relative to the first device support 168 as illustrated by reference points A and B located along the length of the first device support 168. When the first device module 160 is at the second position 166, reference point A and reference point B are located off axis and proximal to the first device module 160. As the first device module 160 moves proximally (retracts) along the first device support 168, the first device support 168 remains stationary because the second device module 162 to which it is coupled via the first front fixed point 172 and the first rear fixed point 174 is also stationary. When the first device module 160 is at the first position 164, reference point A and reference point B are the located proximate to the distal end of the first device module 160

FIG. 10 is a top view illustrating reverse translation of a device module linearly relative to a device support in accordance with an embodiment. When the second device module 162 moves backwards away from the patient (as indicated by arrow 169) from a first position 176 to a second position 178, the second front fixed point (not shown) distal to the second device module 162 takes the load developed as a cassette of the second device module 162 (and the device module) moves along the second device support 170 (e.g., friction between the cassette and the second device support 170). Accordingly, the second device support 170 will not buckle between the cassette on the second device module 162 and the second rear fixed point 175. Since the device supports 168 and 170 are each being supported between two known points, the length of each device support does not need to change. As the second device module 162 moves proximally towards the first device module 160 (which is stationary at its first position 164 in this example) the second device module 162 moves relative to the second device support 170. In addition, the first device support 168 (coupled to the second device module 162 via first front 172 and rear 174 fixed points) moves relative to the first device module 160 as illustrated by reference points A and B located along the length of the first device support 168. When the second device module 162 is at the first position 176, reference point A and reference point B are located proximate to the distal end of the first device module 160 as shown in FIG. 7. As the second device module 162 moves proximally (retracts) along the second device support 170, the second device support 170 remains stationary because it is coupled to a more distal device module (not shown) which is stationary in this example. However, the first device support 168 moves proximally with the second device module 162 to which it is coupled via the first front fixed point 172 and the first rear fixed point 174. At the second position 178 of the second device module 162, reference point A and reference point B are located off axis and proximal to the first device module 160.

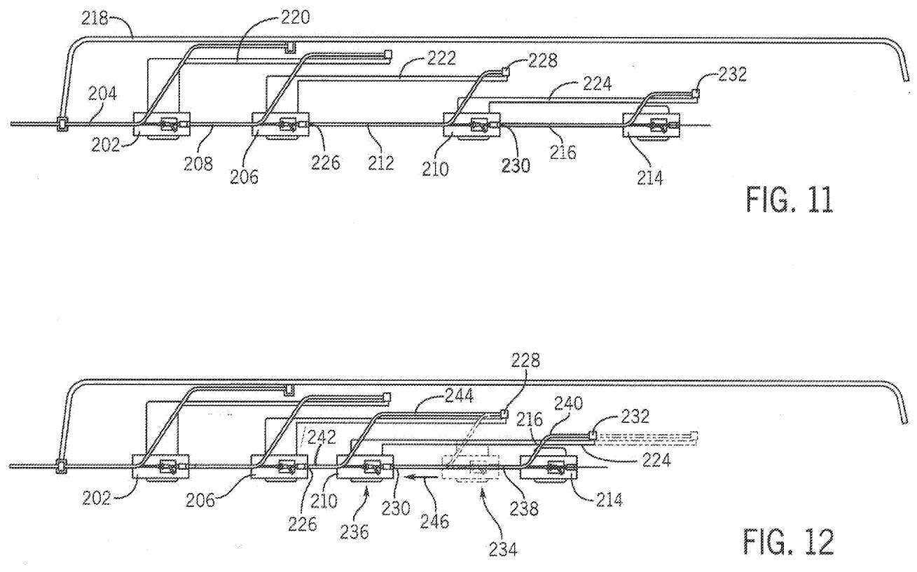

FIG. 11 shows a simplified top view of four device modules and four device supports for a robotic drive in accordance with an embodiment. A first device module 202 includes a first device support 204 with one end connected to a support arm 218 and one end connected to a distal support point. A second device module 206 includes a second device support 208 with one end connected to a support arm 220 and one end connected to the first device module 202. A third device module 210 includes a third device support 212 with one end connected to a first front (or distal) fixed point 226 on the second device module 206 and another end connected to a first rear (or proximal) fixed point 228 on a support arm 222. A fourth device module 214 includes a fourth device support 216 with one end connected to a second front (or distal) fixed point 230 on the third device module 210 and another end connected to a second rear (or proximal) fixed point 232 on a support arm 224. In various embodiments, the support arms 218, 220, 222 and 224 may be connected to the device module or the cassette of a drive module. In another embodiment, the support arms 218, 220, 222 and 224 may be foldable, telescoping or use other methods to shorten the length of the support arm when not in operation. FIG. 12 shows a simplified top view illustrating movement of a device module relative to a device support in accordance with an embodiment. The third device module 210 starts at a first position 234 (shown with dotted lines) and moves to a second position 236 (as indicated by arrow 246). As the third device module 210 moves forward (toward a patient), it moves along the third device support 212 that is fixed to second device module 206 at the first front fixed point 226 and is fixed to the support arm 222 extending from the second device module 206 at the first rear fixed point 228. As the third device module translates, the portion of the device support 212 moving through the third device module 210 changes, while the first front 226 and first rear 228 fixed points do not move. The length of a first section 242 of the device support 212 spanning between the second device module 206 and the third device module 210 decreases while the length a second section 244 of the device support 212 spanning between the third device module 210 and the rear fixed point 228 increases. This allows the third device module 210 (and the associated EMDs) to remain fully supported between the span between the third device module 210 and the second device module 206 during linear motion. Another relative motion occurring during the movement of the third device module 210 between the first position 234 and the second position 236 involves the fourth device support 216 of the fourth device module 214 and the second front (or distal) 230 and second rear (or proximal) 232 fixed points for the fourth device support 216. The fourth device support 214 is fixed to the third device module 210 at the second front fixed point 230 and is fixed to the support arm 224 extending from the third device module 210 at a second rear fixed point 232. Because the third device module 210 is moving, the second front 230 and second rear 232 fixed points are moving as well. A first section 238 of the fourth device support 216 slides through the fourth device module 214, increasing in length in the span between the forth device module 214 and the third device module 210 while a second section 240 of the fourth device support 216 decreases in length in the span between the fourth device module 214 and the rear fixed point 232.

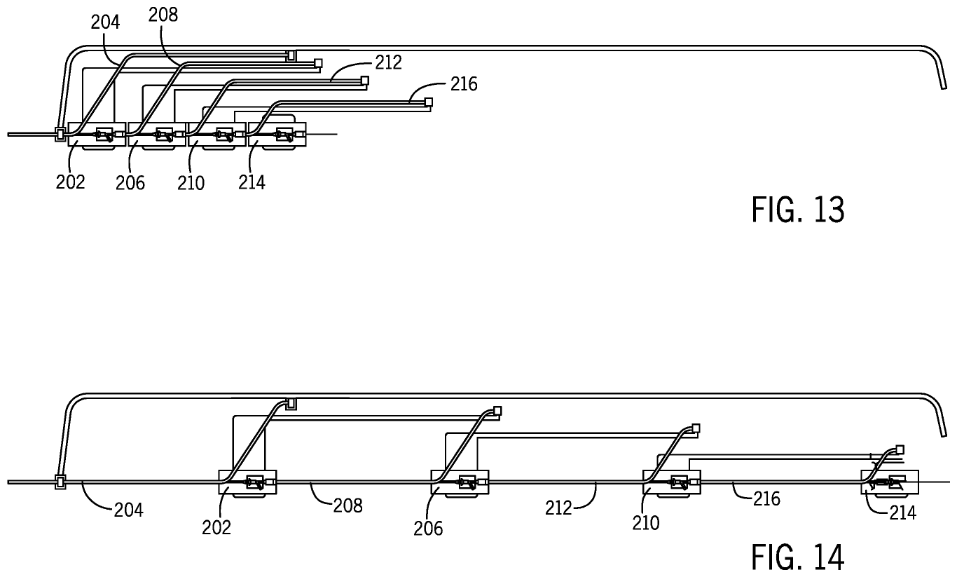

FIG. 13 shows a simplified top view illustrating the four device modules of FIG. 11 in a forward position relative to their respective device support in accordance with an embodiment. In FIG. 13, the first device module 202, the second device module 206, the third device module 210 and the fourth device module 214 are each shown in the maximum forward position along their respective device support 204, 208, 212 and 216. FIG. 14 shows a simplified top view illustrating the four device modules of FIG. 11 in a withdrawn position relative to their respective device support in accordance with an embodiment. In FIG. 14, the first device module 202, the second device module 206, the third device module 210 and the fourth device module 214 are shown in a maximum extended (rear) position along their respective device support 204, 208, 212 and 216. In an embodiment, the device support length is determined by the straight length of the device support and the S-shaped spline that takes the device support off the longitudinal device axis of a device module and directs it towards the support arm longitudinal axis. In one embodiment, each device support 204, 208, 212 and 214 may include compliance to pretention the device support to help with slack when transitioning between forward and reversed directions.

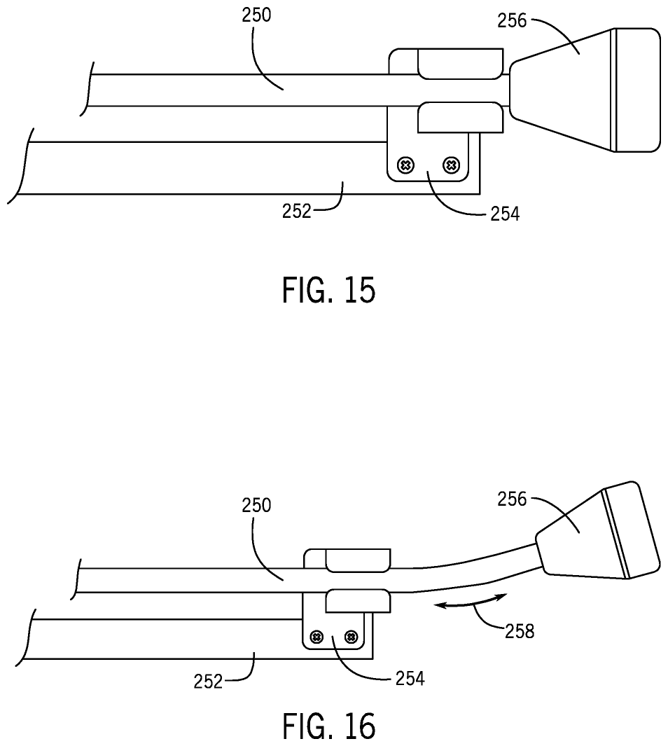

As discussed above, each device support is constrained at a rear (or proximal) fixed point that is connected to a support arm extending from a device module in front (e.g., distal to) the device module associated with the device support. In an embodiment, the rear (or proximal) fixed point includes a rear constraint that may be configured to only react tensile forces. FIG. 15 is a side view of a proximal end of a device support that is extended and a rear constraint for a rear (or proximal) fixed point to which the device support is connected in accordance with an embodiment and FIG. 16 is a side view of a proximal end of a device support that is partially retracted and a rear (or proximal) constraint for a rear fixed point to which the device support is connected in accordance with an embodiment. A proximal end 252 of a support arm includes a retaining clip 254 which holds the proximal end of the device support 250. A hard stop 256 is positioned on the end of the device support and is configured to hold the device support in tension when the device support moves forward and allowing the device support to be retracted for device loading (as described above with respect to FIGS. 5 and 6). Forward motion and retraction of the device support 250 is indicated with arrow 258. An operator may pull back on the device support 250 without removing it from the retaining clip 254. The rear constraint formed from the retaining clip 254 and the hard stop 256 only reacts tensile forces. The device support will not buckle because the retaining clip 254 cannot react compressive forces.

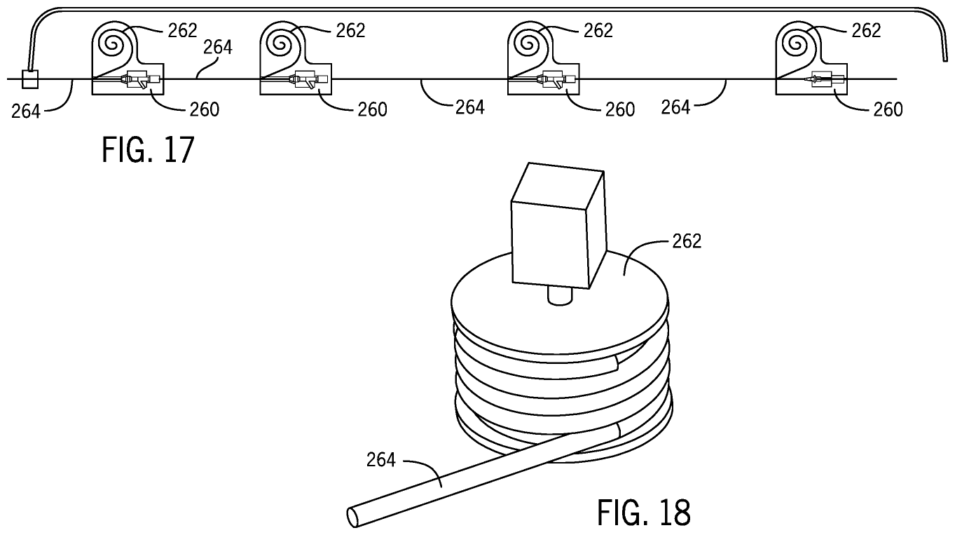

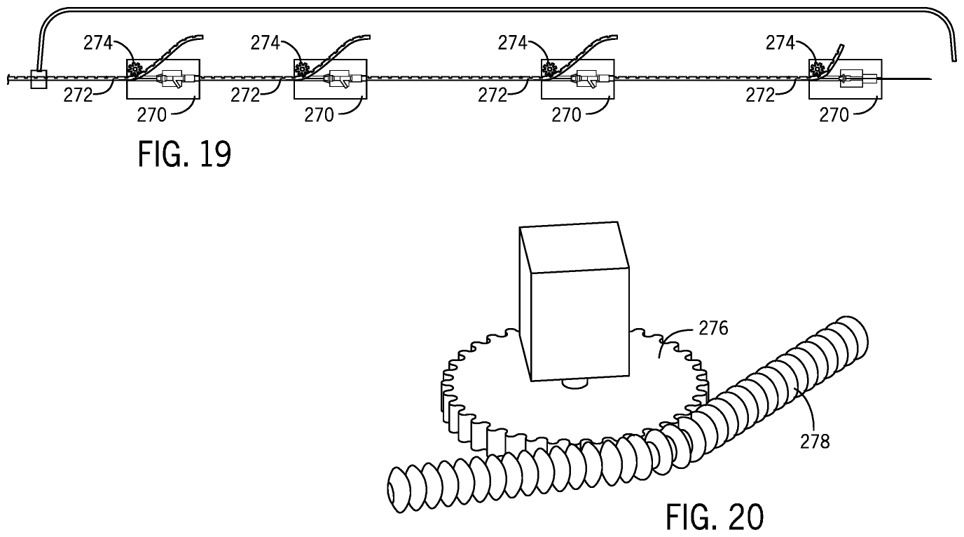

In another embodiment, the tension on the device support provided by front (or distal) fixed point that connects the device support to a more distal device module and a rear (or proximal) fixed point is created by storing the proximal end of the device support on a reel or spool at each cassette. In this embodiment, support arms would not be required to provide the fixed point on the proximal end of the device support. FIG. 17 shows a simplified top view of device modules with device supports stored on a reel in accordance with an embodiment and FIG. 18 shows an exemplary spooled tensioner in accordance with an embodiment. In FIG. 17, each device module 260 includes a reel or spool 262 on which the device support may be wound. An exemplary spooled tensioner is shown in FIG. 18 that includes a spool 262 on which the flexible tube of the device support 264 is wound. The proximal end of the device support is fixed to the spool 262. The distal or “free” end of the device support may be pulled out by an operator or robotically actuated by the robotic drive and attached to a front fixed point on a distal cassette. A torque may be applied to the spool to apply tension to the device support 264. The torque could be applied by a solely mechanical apparatus such as a constant torque spring or a rack and pinion. In another embodiment, the torque may be applied by, for example, a motor (not shown) which is controlled by the control computing system 34 (show in FIG. 2). FIG. 19 shows a simplified top view of device modules with driven device supports in accordance with an embodiment and FIG. 20 shows an exemplary geared tensioner in accordance with an embodiment. In FIG. 19, each device module 270 includes a drive mechanism 274 which interacts with or engage a device support 272 to provide tension on the device support and allow the device support 272 to move forward and backwards. The drive mechanism may be, for example, a wheel or gear. In one embodiment, the drive mechanism 274 may engage the device support via friction on the walls of the flexible tube of the device support 272. In another embodiment, the device support may have radial holes along a side which are then engaged by a pin-drive gear, also called a tractor feed. In another embodiment, the device support is a ribbed or convoluted tube and the drive mechanism is a toothed gear that engages and tensions the ribbed or convoluted tube. An exemplary geared tensioner 276 is shown in FIG. 20 that engages a convoluted flexible tube 278.

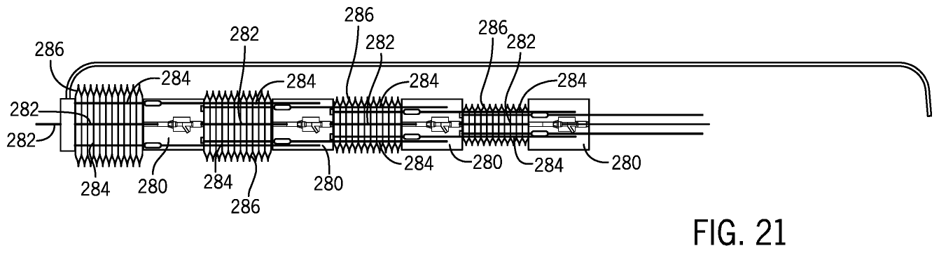

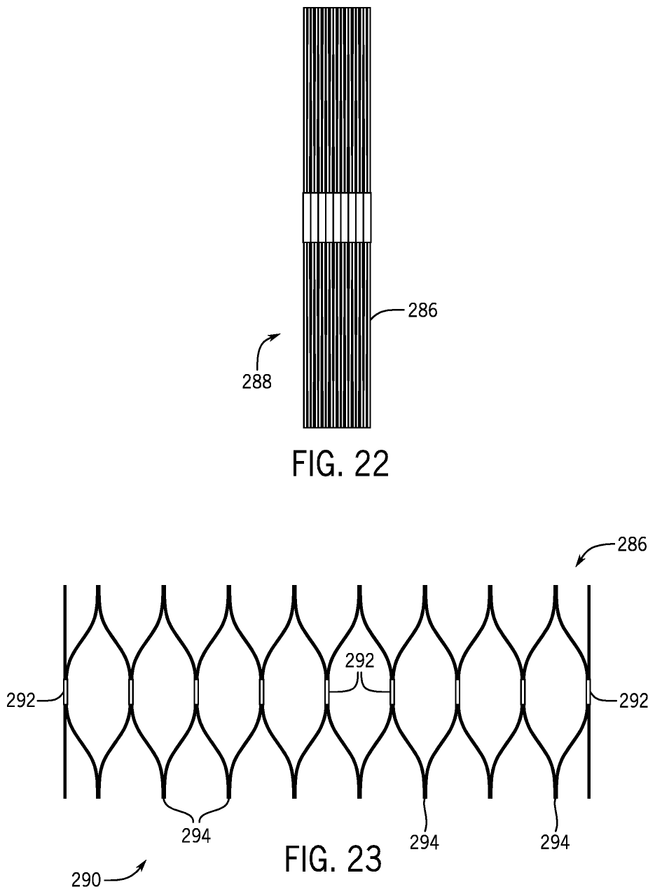

In another embodiment, the device support may be an accordion or spring. FIG. 21 shows a simplified top view of device modules with device supports formed with accordions or springs in accordance with an embodiment. In FIG. 21, a device support between the device modules 280 is formed from an accordion element 286 and two linear guides 284 which are positioned in parallel to one another on opposite sides of the accordion element 286. An EMD 282 is positioned through openings 292 (shown in FIG. 23) in each segment 294 (shown in FIG. 23) of the accordion element 286. The accordion based device support is always in tension. In one embodiment, the accordion device support has compliance built in such that is capable of handling the relative translational motion between two device modules 280. Even though the accordion member acts as a tensile spring and typically stays in tension, it may still deflect from the device axis when axial load is applied. The linear guides (or guiding rails) 284 shown in FIG. 21 constrain the accordion so it is limited in deflection away from the device axis. In one embodiment, the linear guides 284 of a first device module mount to the proximal end of the more distal second device module and the other end of the linear guides 284 are free to slide through the accordion and the first device module. An embodiment where four accordions exist to support four device modules can have the accordion linear guides offset so that the linear guides for not interfere with one another when the device module are close. FIG. 22 illustrates a compressed state 288 of the accordion element 286. The linear guides are not shown in FIG. 22 for clarity. FIG. 23 illustrates a stretched state 280 of the accordion element 286. The linear guides are not shown for clarity. The accordion element 286 includes multiple segments 294 that each include an opening 292 through which an EMD may be positioned. The number of segments 294 and the lengths of the segments 294 may be optimized so that the unsupported distance between discrete segments 294 is such that an EMD will not buckle at maximum loads experienced during a procedure. The accordion device support has multiple flexures which auto-balance to give equal spacing regardless of the overall tension so that no single gap across the length of a segment 294 becomes large enough for buckling. In other words, the gaps across each segment 294 length want to be the same across all segments 294. This helps minimize the unsupported distance an EMD needs to travel, which allows the accordion element 286 to reach higher loads before buckling.

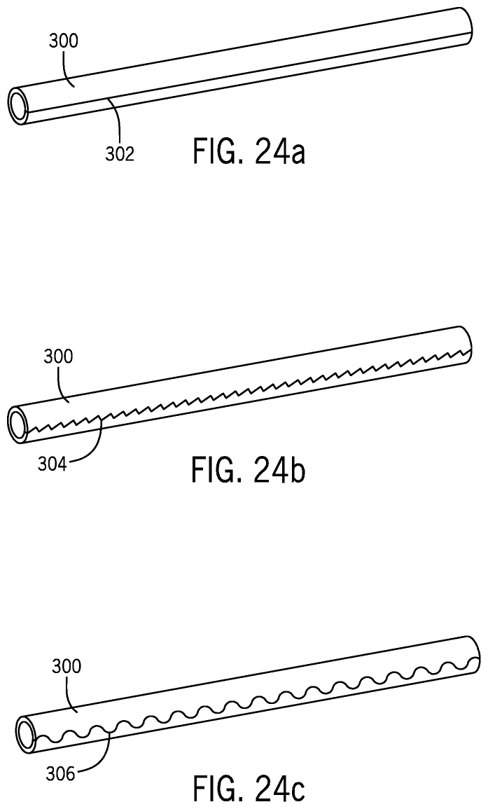

The profile of a device support formed from a flexible tube should support being opened and closed, for example, to allow EMDs to be loaded into the device support. When the slit at the distal end of the device support flexible tube is forced apart (e.g., using a splitter as discussed further below, the device support may be advanced to encapsulate the EMD and when, closed, the EMD is adequately supported and retained so as to not pop out and buckle. FIGS. 24 (a)-(c) are perspective views of exemplary slit shapes for a device support flexible tube in accordance with an embodiment. In FIG. 24(a), a device support flexible tube 300 is shown with a straight slit 302 lengthwise along the tube. In another example, a device support flexible tube 300 may have a serrated shaped slit 304 lengthwise along the tube as shown in FIG. 24(b). In yet another example, a device support flexible tube 300 may have a wave shaped slit 306, similar to a sine wave, lengthwise along the tube as shown in FIG. 24(c). The slit of the device support 300 may be opened by a wedge or splitter (shown in FIGS. 27-29 and discussed further below) that is positioned close to an entry point for an EMD to the device support. The wedge or splitter spreads the opening wide enough to clear the EMD. The elasticity of the flexible tube causes the slit to recover and close on the other side of the EMD, encapsulating and retaining the EMD. The serrated shape and shape similar to a sine may be used so that the material in the area of the slit overlaps so as to improve EMD retention in the device support.

The EMDs utilized in a robotic drive for an interventional procedure may vary in size, for example, the various EMDs that may be used may vary from 9FR to 2FR or even a 0.010″ guidewire. For example, in a multi-axial robotic drive configured for an endovascular therapy procedure treat acute ischemic stroke, it can be expected that the first EMD in the device stack-up is between 6 and 9 FR. The second and third EMDs in the device stack-up may be between 2.5 to 6 FR. The fourth EMD may be a wire-based EMD with a diameter between 0.010″ to 0.038″. In order to properly support and retain EMDs with different sizes, different device supports may be provided for each EMD where the device support for each EMD is designed to work with the corresponding size of EMD. For example, by minimizing the diametrical clearance between the EMD and the device support tube, any device buckling inside the tube will store less energy and have less linear motion hysteresis. In an embodiment, the device support of each cassette may be designed to be modular so that the correctly sized device support may be added to a cassette based on the EMD being supported by the cassette. In addition, a splitter and device support connector (both discussed further below with respect to FIGS. 27-29) that are designed to work with a specific size of EMD may also be modular and switched based on the specific size of EMD being supported by a cassette. In another embodiment, different versions of a cassette may be provided for each subset of device sizes, where the cassette has an appropriately sized device support pre-installed. The appropriate cassette design for the specific size or range of sizes of an EMD may be mounted to a drive of the robotic drive and removed when a different design is needed for a different size of EMD or a different size range. For example, a cassette may be designed to support a range of sizes of the wire-based EMD which can vary between 0.010″ and 0.38″.

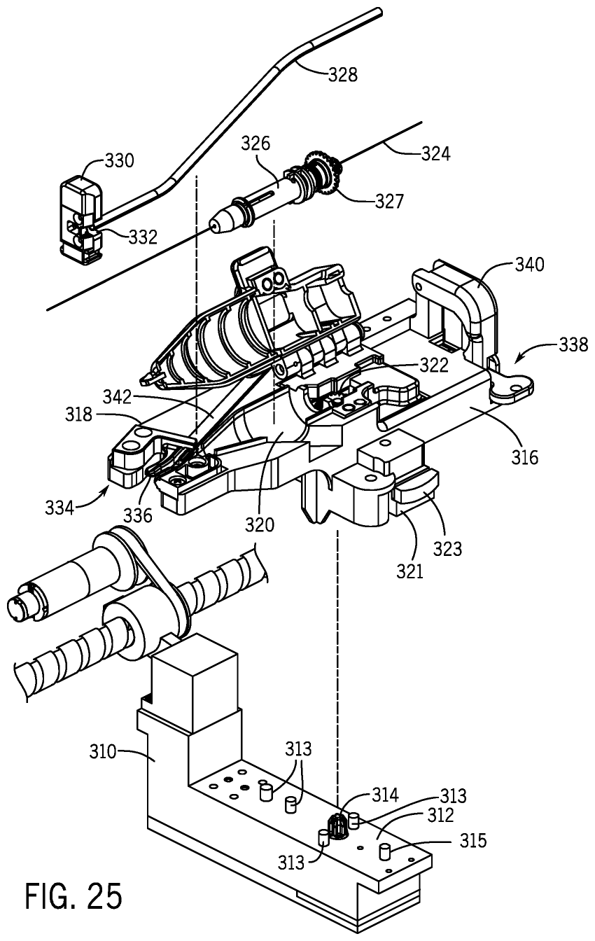

As discussed above with respect to FIG. 3, a device module 32 of a robotic drive 24 includes a drive module 68 and a cassette 66 mounted on and releasably coupled to the drive module 68. FIG. 25 is an exploded view of a device module and an elongated medical device in accordance with an embodiment. A drive module 310 includes a mounting surface 312 and a coupler 314. A motor and a drive belt (not shown) may be housed in the drive module 310 and connected to the coupler 314. The motor and belt are used to control a rotational position of the coupler 314. Drive module 310 may include an encoder (not shown) for device position feedback. The drive module 310 shown in FIG. 25 has one coupler 314, however, it should be understood that the drive module 310 may have more than one coupler 314 and more than one motor. (for example, one motor for each coupler or one motor driving multiple couplers) The rotation of the coupler 314 may be used to provide another degree of freedom for an EMD positioned in a cassette 316 that may be mounted on the mounting surface 312 so as to interface with the coupler 314. For example, the coupler 314 may be used to rotate an EMD 324 when the EMD is positioned in the cassette 316. If the drive module 310 has two or more couplers 314, each coupler may be used to provide a degree of freedom for an EMD.

As mentioned, a cassette 316 may be positioned on the mounting surface 312 of the drive module 310 and used to interface with an EMD 324 positioned in the cassette 316. The cassette 316 includes a housing 318. In an embodiment, the cassette housing 318 may be releasably attached to the drive module 310. The drive module 310 may also include one or more additional elements 313 on the mounting surface 312 such as, for example, positioning pins, alignment pins, etc. to interact with elements on a cassette 316 (e.g., connection points, slots, channels, etc.) to enable a releasable attachment of the cassette 316 to the drive module 310. In one embodiment, cassette housing 318 is releasably connected to the drive module 310 using a quick release mechanism 321. In one embodiment, the quick release mechanism 321 includes a spring-biased member in cassette housing 318 that is actuated by a latch release 323 that releasably engages with a quick release locking pin 315 secured to the drive module 1010.

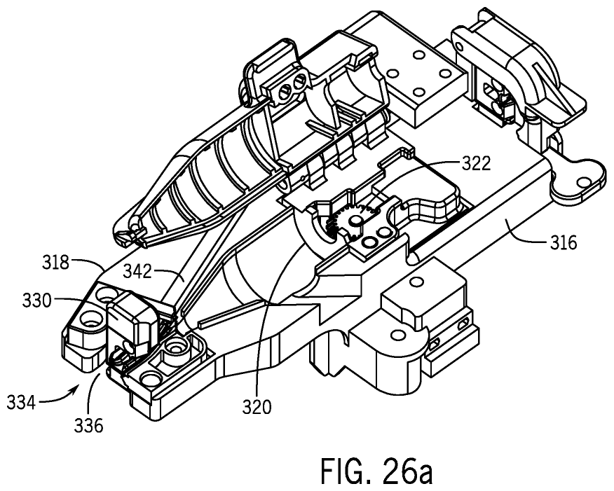

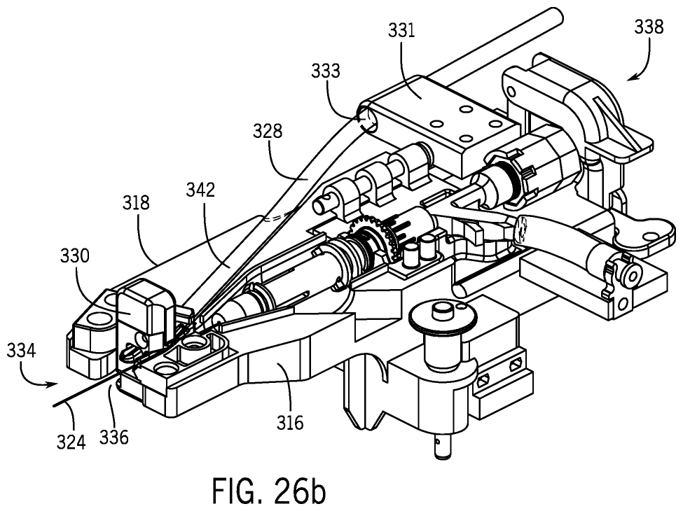

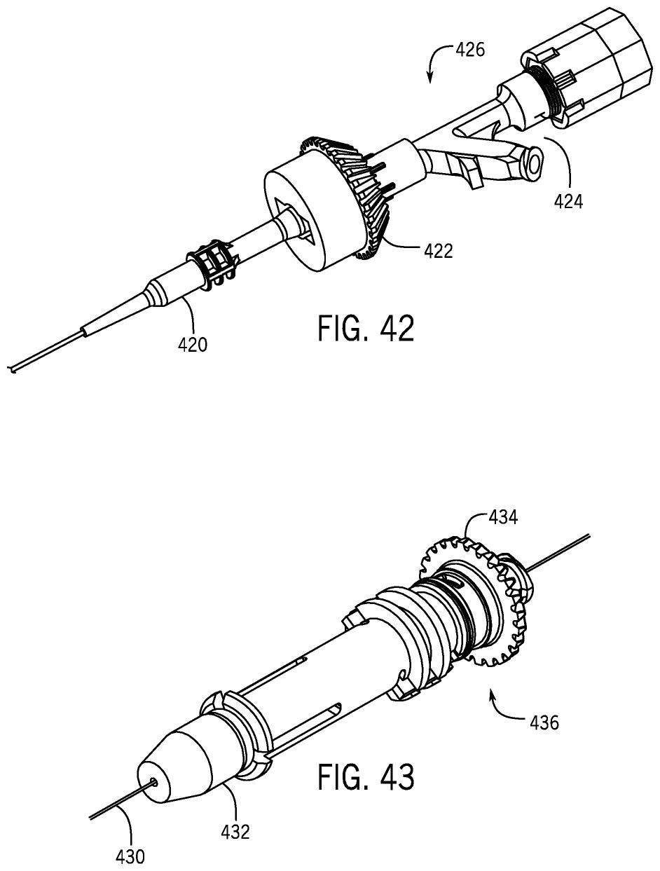

The cassette housing 318 includes a cradle 320 configured to receive the EMD 324. A bevel gear 322 is used to interface with the coupler 314 of the drive module 310 and to interface with the EMD 324 to rotate the EMD 324. In one embodiment, EMD 324 is provided with an on-device adapter 326 (discussed further below with respect to FIGS. 42-44) to interface the EMD 324 to the cassette 316, for example, an interface to bevel gear 322. In the example shown in FIG. 25, the EMD is a guidewire and the on-device adapter 326 is a collet with a gear 327. When power is transferred from the device module 310 to the gear 322 in the cassette 316 (e.g., via the coupler 314), the gear 322 in the cassette interacts with the gear 327 on the collet to rotate the guidewire 324. A device support 328 is positioned in the cassette in a channel 342 which may be covered by the housing 318. As discussed above, the device support 328 and the cassette 316 are configured to move relative to one another. The device support 328 includes a connector 330 which is used to connect to a device module (e.g., to a cassette, to other elements of the device module, or to elements positioned in the device module) distal (or in front of) the cassette 316 in a robotic drive. Connector 330 includes a recess 332. In a withdrawn or retracted position, the connector 330 is positioned in a recess 336 in the housing 318 on a distal end 334 of the cassette 316. As discussed above, the connector 330 and device support 328 may be pulled outward from the cassette 316 so the connector may be attached to a more distal device module (e.g., a cassette of the device module) in the robotic drive. In one embodiment, a forward constraint 340 is provided on a proximal end 338 of the cassette 316 and is used to connect to a connector of a device support on another cassette proximal to (or behind) the cassette 316 in a robotic drive. FIG. 26a is a perspective view of a cassette with a device support installed and in a retracted position in accordance with an embodiment. In the retraced position, the connector 330 is positioned in the recess 336 in the housing 318 at the distal end 334 of the cassette 316. FIG. 26b is a perspective view of a cassette with a device support installed and in a retracted position in accordance with an embodiment. The device support 328 is positioned in a channel 342 of the cassette. The cassette 316 incudes a proximal support member 331 positioned on the proximal end 338 of the cassette 316. The proximal support member 331 includes an opening and is configured to provide support to the device support 328. Device support 328 is positioned in and passes through the opening 333. The opening 333 is sized so that the device support can move through the opening 333 as the device support 328 is advanced and retracted.

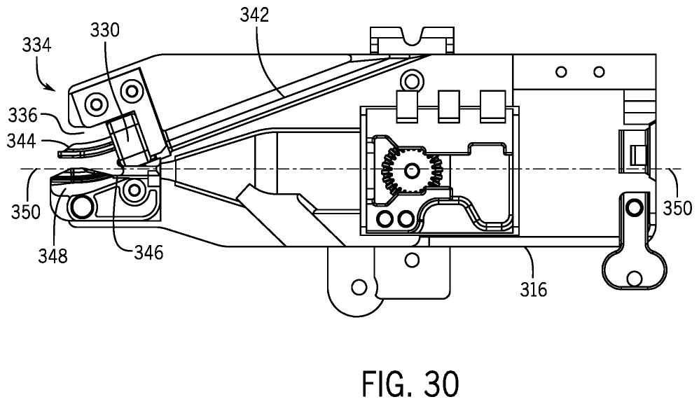

FIG. 27 is a top view of a device support and connector extended from a cassette ahead of an EMD entry point in accordance with an embodiment. A device support 328 and connector 330 are extended out from the recess in the distal end 334 of the cassette housing. A guide 344 and a splitter 348 are positioned in the recess 336 on opposite sides of the path of the device support 328 as it is moved into and out of the recess 336 and channel 342. In the extended position, the device support encapsulates an EMD 324. The EMD enters the device support 328 at an EMD entry point 346 which is located between a proximal section and a distal section of the splitter 348. The proximal and distal sections of the splitter are shown with dotted lines. As mentioned above, the device support 328 includes a lengthwise slit so the device support may be forced apart (e.g., by using splitter as described below) and closed to allow the device support to encapsulate an EMD as the device support is advanced. The connector 330 holds open an end of the device support tube allowing it to pass over the splitter 348 as shown in FIG. 29. Referring to FIGS. 27 and 29, the splitter 348 holds the slit in the device support 328 open as the EMD 324 is encapsulated by the device support 328 as the connector 330 and device support 328 pass over the splitter 348 and EMD entry point 346. The end of the device support tube 328 is positioned in a recess 332 of the connector 330. Using the splitter 348 to hold open the device support 328 on both sides of EMD entry point 346 reduces or eliminates friction forces on the EMD 324. For example, this prevents the walls of the device support 328 tube from rubbing the EMD 324 which can cause damage to the EMD 324 at the entry point 346 and would introduce noise to a load sensing system (not shown) which may be used to read the force or torque the EMD is subjected to. The EMD 324 passes through a cavity 352 in the center of the splitter 348. The connector 330 and the splitter 348 are designed so that the device support 328 is held open as it passes over a gap between the proximal and distal section of the splitter 348. Splitter 348 is also designed such that the unsupported length of the EMD 324 at any point is not such that it can catastrophically buckle. Guide 344 is configured to guide the device support 328 over the gap and retain the device support 328 on the splitter 348. As mentioned above, the splitter 348 may be designed for specific EMD and device support size ranges. FIG. 28 is a top view of a device support and connector withdrawn behind an EMD entry point in accordance with an embodiment and FIG. 30 is a top view of cassette with a device support connector withdrawn and off of a device axis to facilitate loading of an EMD in accordance with an embodiment. To facilitate loading of an EMD 324 in a cassette 316 (shown in FIG. 25), the device support 328 and connector 330 are retracted into the recess 336 before an EMD 324 is loaded. As shown in FIGS. 28 and 30, the connector 330 may be retracted onto the splitter 348 and guide 344 and behind (or proximal to) the EMD entry point 346. In addition, the retracted (or withdrawn) position of the connector 330 is off of a longitudinal EMD axis 350. This allows for EMD placement into cassette 316, for example, loading a side loading EMD. Retracting the connector 330 behind the EMD entry point also reduces the unsupported EMD length and reduces working length loss.

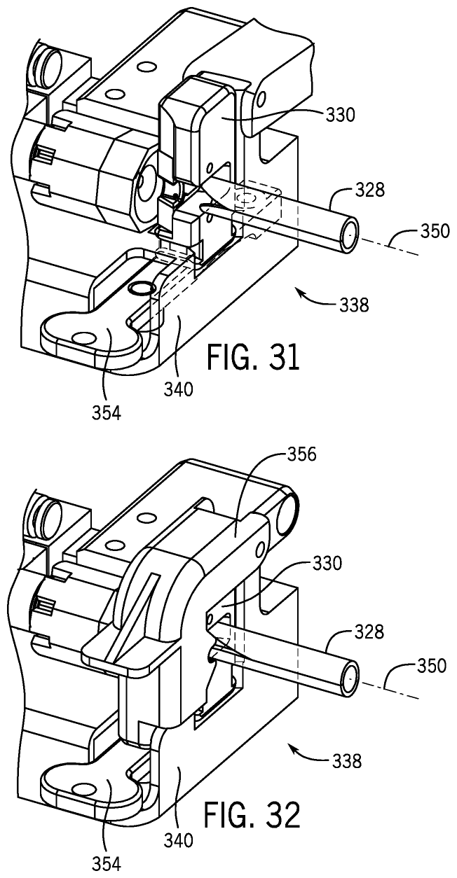

As discussed above, the connector 330 and device support 328 may be pulled outward from the cassette 316 so the connector may be attached to a more distal device module (e.g., a cassette of the device module) in the robotic drive. In an embodiment, a forward constraint 340 (shown in FIG. 25) may be provided on a proximal end 338 of a first cassette and is used to connect to a connecter of a device support on a second cassette proximal to (or behind) the first cassette in the robotic drive. FIG. 31 is a perspective view of a forward constraint and a connector in accordance with an embodiment. Forward constraint 340 includes a latching mechanism 354, for example, a spring latch. A connector 330 of a device support 328 from a proximal cassette (not shown) is attached to the spring latch 354. In one embodiment, the connector 330 connects to the latching mechanism 354 by pushing the connector 330 into the foreword constraint 340. In an embodiment, the latching mechanism 354 may require no secondary motion other than axial translation to engage the latching mechanism 354, but may require one or more additional movements to disengage the latching mechanism 354 and remove the connector from the forward constraint 340. For example, there may be buttons, levers or knobs which may need to be released before the connector 330 becomes disengaged. The connector 330 may be manually disengaged or disengaged using a control computing system 34 (shown in FIG. 2). The connector 330 attaches to the forward constraint 340 approximately along the longitudinal EMD axis 350 of an EMD (not shown) contained in the device support 328. This prevents shearing of the EMD by moving perpendicular to the latching mechanism 354. In another embodiment, a secondary latch or tightening mechanism may be provide to further secure the connector 330 and reduce play. FIG. 32 is a perspective view of a forward constraint with a lid in accordance with an embodiment. In FIG. 32, a lid 356 is connected to the forward constraint 340, for example using a pivot. The lid 356 may be closed over the connector 330 and latched to further constrain the connector 330 in the forward constraint 340.

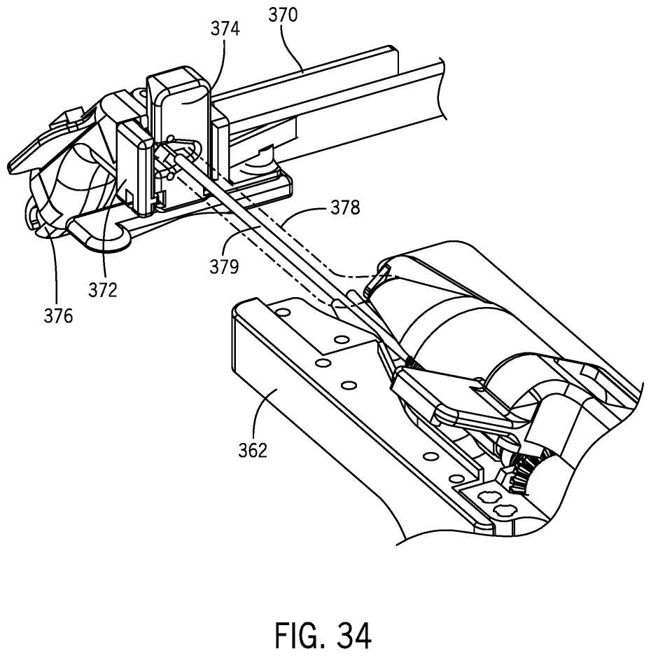

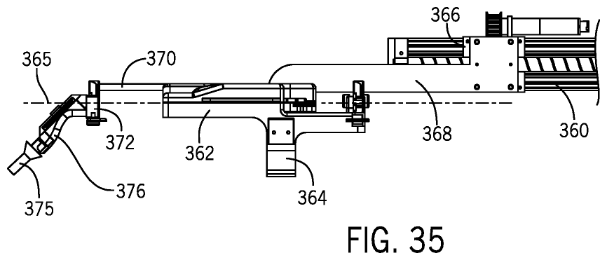

As discussed above with respect to FIG. 4, a distal support connection mounted to a distal support arm may be used to provide a front (or distal) fixed point to support the distal end of the device support in the cassette of the most distal device module in the robotic drive, i.e., the device module closest to the patient. FIG. 33 is a perspective view of a distal support arm and distal support connection in accordance with an embodiment. A cassette 362 is mounted to a drive module 364 which is connected to a stage 366 using an offset bracket 368. The stage 366 is movably mounted to a rail or linear member 360 and may be moved linearly along the rail 360. A distal support arm 370 may be attached to a frame of the robotic drive, for example, a frame of the rail 360. In one embodiment, the distal support arm 370 may be rigidly attached to the frame. In another embodiment, the distal support arm 370 may be attached to a patient table or the patient. The distal support arm 370 extends away from the robotic drive and is connected to a device support connection 372 to provide a distal fixed point for the device support at an introduced sheath hub. In one embodiment, the distal support arm 370 may also be used to provide a distal define for the cassette 362 and drive module 364. A distal define is used to define the most distal aspect of the most distal device (e.g., cassette 362 and drive module 364) of the robotic drive. In another embodiment, the distal define may be provided using a separate distal define arm (not shown) that may be coupled to, for example, the frame of the robotic drive. The distal support connection 372 may also be coupled to an introducer sheath hub. An introducer interface support 376 may be connected to the device support connection 372. A connector 374, for example, a connector on a distal end of a device support as described above with respect to FIGS. 27-30 may be attached to the device support connection 372 to provide a front (or distal) fixed point and support for the distal end of the device support. A device support is not shown in FIG. 33, but would be positioned in by the cassette 362 as shown in FIG. 34. FIG. 34 is a perspective view of a distal support connection coupled to a device support and connector in accordance with an embodiment. A device support 378 is shown as a dotted line encapsulating an EMD 379 and extending between the cassette 362 and the device support connection 372. The connector 374 is attached to the device support connection 372. The device support connection 372 may be, for example, a forward constraint such as described above with respect to FIGS. 31 and 32. The device support connection 1072 is mounted to a distal support arm 370 and may be connected to an introducer interface support 376. FIG. 35 is a side view of a distal support arm, distal support connection and an introducer interface support in accordance with an embodiment. The introducer interface support 376 is configured to support an EMD 379 (shown in FIG. 34) between the device support 378 (shown in FIG. 34) and an introducer sheath 375 connected to a distal end of the introducer interface support 376 as discussed further below. The introducer interface support 376 ensures that the EMD 379 does not buckle or prolapse between the distal end of the device support 378 and the hub of an introducer sheath 375. In an embodiment, the introducer interface support 376 may also be used to redirect an EMD from a position that is axially aligned with the robotic drive device axis 365 to a position that is axially aligned with the introducer sheath 375 or other supporting member.

The introducer sheath 375 is inserted at an access point (e.g., the femoral artery) into a patient's vasculature that will lead the EMD to the target location in the patient (e.g., a lesion). The introducer sheath 375 should be held in place so that it does not come out of the patient. In one embodiment, the distal support arm 370 and the device support connection 372 may be used to fix the position of the introducer sheath 375 and may react forces on the introducer sheath 375 created from the friction between the introducer sheath 375 and the EMD moving inside of the introducer sheath 375. In another embodiment the introducer sheath 375 may be supported by a separate structure than the distal support arm 370 and device support connection 372, for example, the introducer sheath 375 may be attached to the patient or a patient table using known methods.

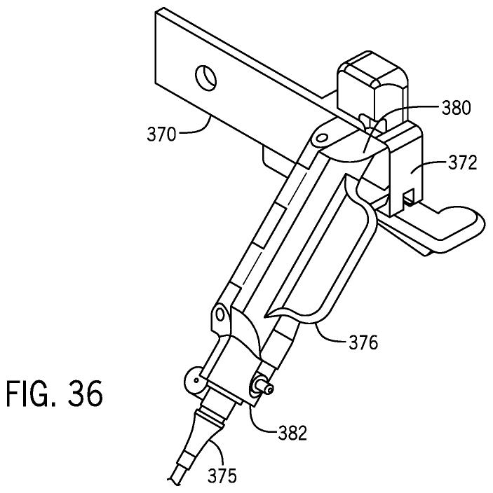

FIG. 36 is a perspective view of an introducer interface support connected to an introducer sheath in accordance with an embodiment. The introducer interface support 376 is connected at its proximal end 380 to a device support connection 372 that is connected to a distal support arm 370. An introducer sheath 375 is connected to a distal end 382 of the introducer interface support 376. The introducer interface support 376 may be configured to receive the introducer sheath 375 with a side port (not shown). The side port and its tubing (not shown) can allow for administration of medicine, contrast or saline injection or drawing blood samples. An EMD (not shown) enters the body of a patient through the introducer sheath 375 which is inserted into a vessel (typically an artery). In one embodiment, the introducer interface support 376 opens to allow the EMD to be placed in the introducer interface support 376. In another embodiment, an EMD may be inserted axially into the introducer interface support 376. In another embodiment, the EMD and introducer interface support 376 may be frictionally fit so that the introducer interface support 376 does not need to open or have the EMD inserted axially. As mentioned above, the introducer interface support 376 may be configured to redirect an EMD from a position that is axially aligned with the robotic drive device axis 365 (shown in FIG. 35) to a position that is axially aligned with the introducer sheath 375 or other supporting member. The introducer interface support 376 also provides support to the EMD in the distance between the connector 372 and the introducer sheath 375. The introducer interface support 376 may be rigid (as shown in FIG. 36) or flexible. For example, the introducer interface support 376 may be made of flexible material or the introducer interface support 376 may have a joint near the device support connection 372 which allows for a limited range of motion of the distal end 382 (where the introducer sheath 375 is held) to account for perturbation of the robotic drive or movement of the patient.

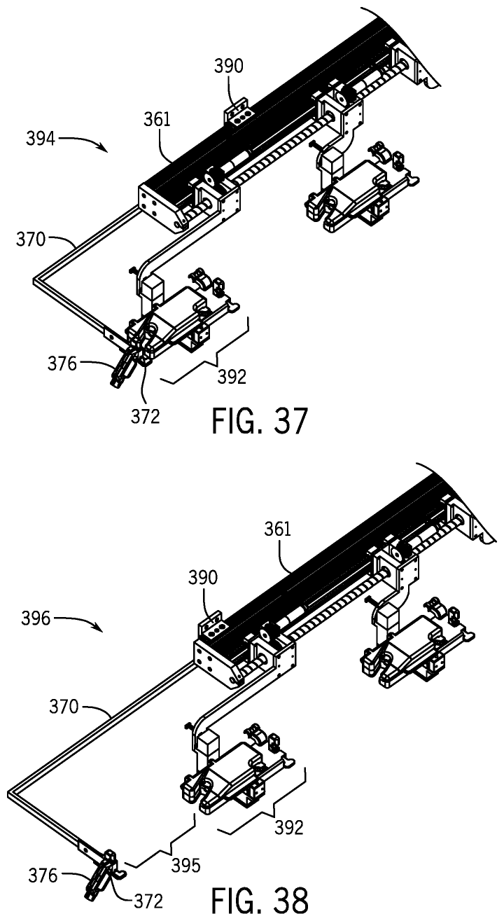

In another embodiment, the distal support arm 370 may be movably connected to the robotic drive. A moveable distal support arm 370 may have one or more degrees of freedom to account for excess exposed EMD length that may not need to be actuated. For example, with shorter patients and/or less tortuously, more of the first guide catheter may be exposed because it will never need to enter the patient. If the distal support arm (and therefore the device support connection 372) can move forward, it can account for the excess length of the guide catheter that does not need to be actuated. This may also help reduce the overall length of the rail or linear member 361 (and rail 360 shown in FIGS. 33 and 35). FIG. 37 is a perspective view of a movable distal support arm in a first position in accordance with an embodiment. A distal support arm 370 may be moveable connected to a rail or linear member 361 using a stage 390. In FIG. 37, the distal support arm 370 is in a first position 394 where the distal support connection 372 is located proximate to with the distal end of a device module 392. The stage 390 may be manually or robotically moved along the rail 361 to change the position of the distal support arm 370. FIG. 38 is a perspective view of a moveable distal support arm in a second position in accordance with an embodiment. In FIG. 38, the stage 390 and the distal support arm 370 have been moved linearly to a second more distal position 396 from the device module 392. Accordingly, the device support connection 372 and the device module 392 are separated by a distance 395. In the embodiment shown in FIGS. 37 and 38, the distal support arm 370 has one degree of freedom. In another embodiment, the distal support arm 370 may be an articulating or driven arm with multiple degrees of freedom.

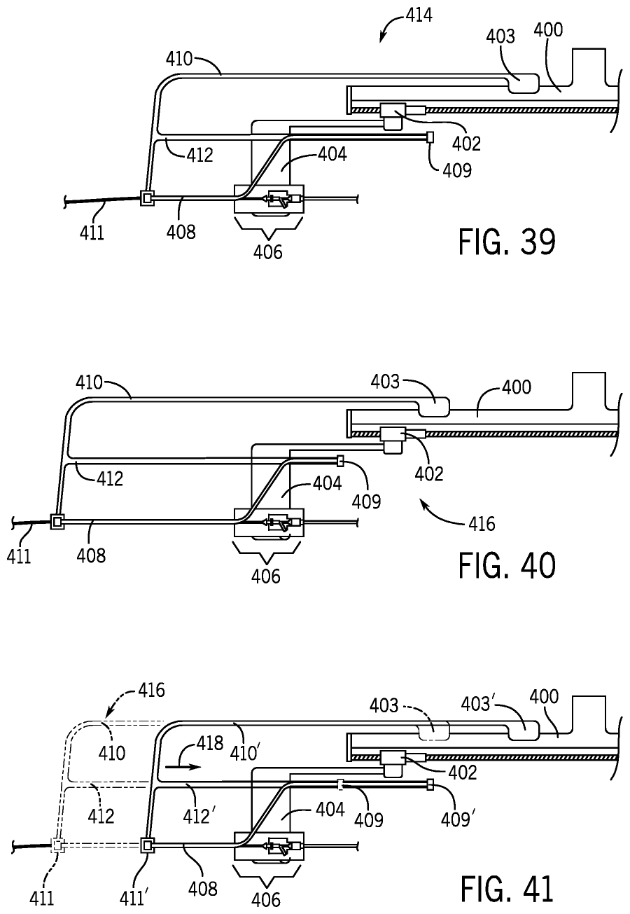

As discussed above, each end of the device support may be connected to fixed point (front (or distal) and rear (or proximal)) to provide appropriate tension to the device support between device modules or between most distal device module and a device support connection to prevent an EMD from buckling. The device support connection 372 described above provides a front (or distal) fixed point for the device support of the most distal cassette in the robotic drive. The device support of the most distal cassette may be provided with a rear (or proximal) fixed point using a support arm (e.g., support arm 118 shown in FIG. 4) that is connected to the distal support arm 370. For a moveable distal support arm, the support arm will also be moveable. FIG. 39 is a top view of a moveable distal support arm and movable support arm in a first position in accordance with an embodiment. In FIG. 39, a distal support arm 410 is in a first position 414. A device module 406 is connected to a rail or linear member 400 using a first stage 402. A device support 408 is positioned in the device module 406 (e.g., in a cassette of the device module) and a distal end of the device support 408 is connected to a device connection point 411 (front (or distal) fixed point) connected to the distal support arm 410. A proximal end of the device support 408 is connected to a proximal end of a support arm 412 at a rear (or proximal) fixed point 409. A second stage 403 is connected to the rail 400 (or a different rail (not shown) in the system) and may be manually or robotically moved along the rail 400 to change the position of the distal support arm 410 and the support arm 412. FIG. 40 is a top view of a moveable distal support arm and movable support arm in a second position in accordance with an embodiment. In FIG. 40, the second stage 403, the distal support arm 410 and the support arm 412 have been moved linearly to a second more distal position 416 from the device module 406. The support arm 412 moves with the device support connection 411 so there is always the same length of the device support 408 between the device support connection 411 and the rear fixed point 409. FIG. 41 is a top view illustrating movement of a distal support arm and a support arm from the second position to the first position in accordance with an embodiment. In FIG. 41, the device support connection 411, the support arm 412, the distal support arm 410 and the second stage 403 start at the second position 416 (indicated by dotted lines). The second stage 403 may be actuated to move linearly along the rail 400 to the first position 414 as indicated by arrow 418. The first position of the device support connection, the support arm, the distal support arm, rear fixed point, and the second stage are indicated by the reference numbers 411′, 412′, 410′, 409′, and 403′, respectively.

FIG. 42 is a perspective view of a catheter with an on-device adapter in accordance with an embodiment and FIG. 43 is a perspective view of a guidewire with an on-device adapter in accordance with an embodiment. As used herein, an on-device adapter is a sterile apparatus capable of releasably clamping to an EMD to provide a driving interface. In FIG. 42, a catheter 420 includes a hemostasis valve or hub (e.g., a rotating hemostasis valve) 424 on the proximal end 426 of the catheter 420. An on-device adapter 422 is positioned on the catheter 420 distal to the hemostasis value 424 on the proximal end 426 of the catheter. In the embodiment of FIG. 42, the external surface of the on-device adapter is formed as a gear. The gear feature of the on-device adapter 422 is configured to interact with a gear 322 (shown in FIG. 26a) of a cassette, for example, cassette 316 shown in FIG. 26a. When power is transferred from a device module (not shown) to the gear in the cassette (e.g., via a coupler), the gear in the cassette interacts with the gear 422 on the catheter 420 to rotate the catheter. In another embodiment, rotation of the on-device adapter 422 may be configured to pinch/unpinch the catheter 420. In an embodiment, an internal surface of the on-device adapter 422 is firmly attached to a standard luer section of the elongated medical device (e.g., catheter 420). In another embodiment, the internal surface of the on-device adapter is clamped to a lateral surface it the proximal end of the elongated medical device. In another embodiment, the on-device adapter is attached to a cylindrical section (shaft) of the EMD. In yet another embodiment, the on-device adapter is not directly attached to the EMD, by is attached to the EMD via an interface. The power can transfer from the cassette to the on-device adapter in different ways such as, for example, gears (as mentioned above), or friction surface (e.g., tire and roller), belt, pneumatic, or magnetic/electromagnetic coupling.

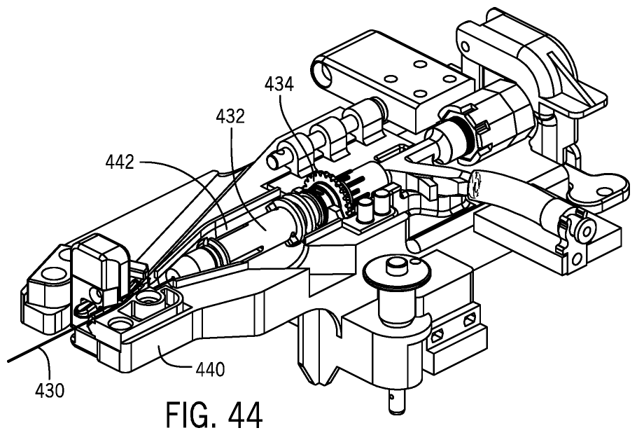

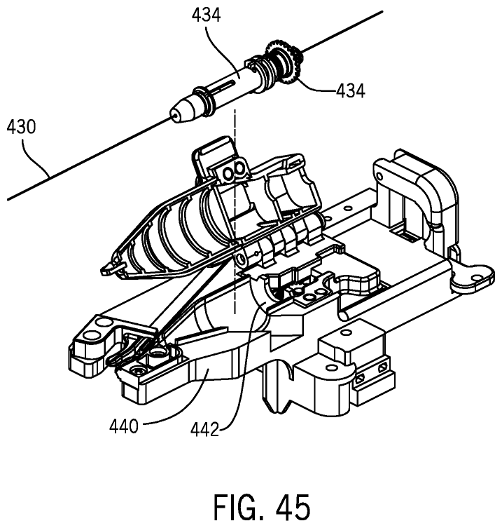

In FIG. 43, a guidewire 430 is shown with an on-device adapter 432. In the embodiment of FIG. 43, the on-device adapter 432 is a collet with a gear 434 on the proximal end 436 of the collet. The collet 432 is configured to grip the guidewire 430. The term collet as used herein is a device to releasably fix a portion of an EMD thereto. In one embodiment the collet includes at least two members that move relative to each other to releasably fix the EMD to at least one of the two members. Fixed means no intentional relative movement of the collet and EMD during operation parameters. The gear 434 is configured to interact with a gear 322 (shown in FIG. 26a) of a cassette, for example, cassette 316 shown in FIG. 26a. When power is transferred from a device module (not shown) to the gear in the cassette (e.g., via a coupler), the gear in the cassette interacts with the gear 434 on the guidewire 430 to rotate the guidewire 430. In another embodiment, rotation of the on-device adapter 432 via gear 436 may be configured to pinch/unpinch the guidewire 430. The elongated medical device and on-device adapter may be positioned in the cassette as shown in FIG. 44. In FIG. 44, a guide wire 430 and collet 432 are positioned in a cradle 442 of the cassette 440. The elongated medical device and the on-device adapter may be removed from one cassette and moved to another unpopulated cassette. FIG. 45 shows a guide wire 430 and collet 432 with a gear 434 removed from the cassette 440. When the cassettes are similar and an on-device adapter is used to interface an elongated medical device to the cassette, the device and on-device adapter may be moved between unpopulated cassettes enabling the number of devices and configuration of the robotic drive to be changed.

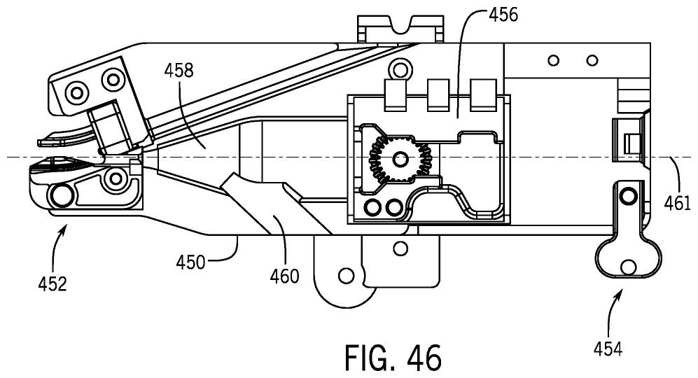

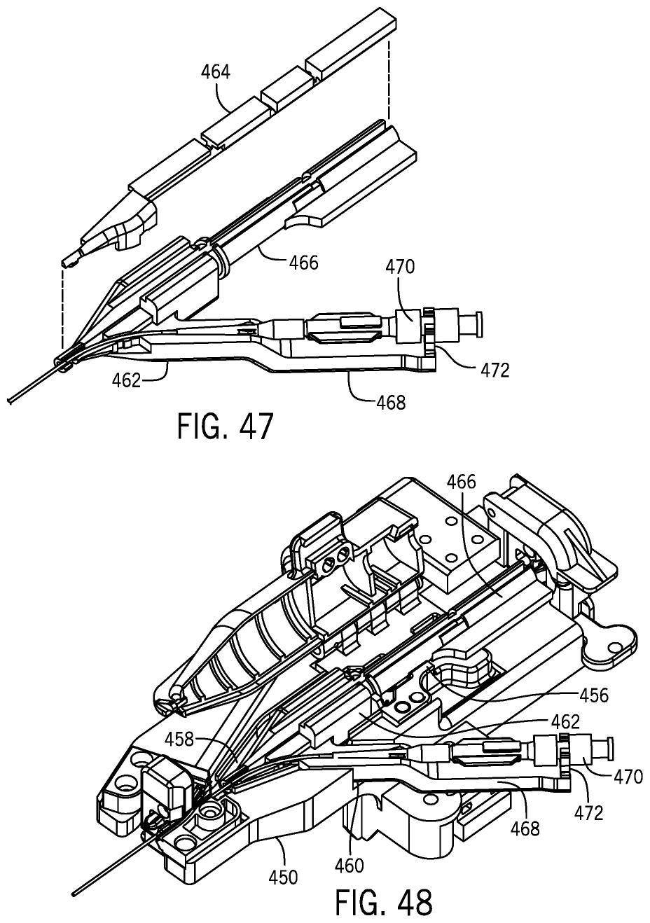

FIG. 46 is a top view of a cassette in accordance with an embodiment. The cassette 450 has a distal end 452 and a proximal end 454 and is typically used to interface with an EMD such as a guidewire or a catheter. The area between the distal end 452 and the proximal end 454 includes a cradle 456, a midsection 458 and an off-axis recess 460 that is positioned angled away from the cassette longitudinal device axis 461. The midsection 458 and off-axis recess 460 may be configured to receive an EMD adapter to interface the cassette with EMDs with atypical proximal ends, for example, a balloon guide catheter (which includes an integrated y-connector) or a rapid exchange device such as a rapid exchange balloon. FIG. 47 is an exploded view of an elongated medical device (EMD) adapter and a lid in accordance with an embodiment. The EMD adapter 462 shown in FIG. 47 is a rapid exchange EMD adapter. The EMD adapter includes a lid 464, a first section 466 and a second section 468. The first section is configured to receive a guidewire. The second section is configured to receive an EMD, e.g. a rapid exchange EMD 470. In one example, the EMD 470 is a rapid exchange balloon. The second section 468 is positioned at an angle from the longitudinal axis of the first section 466. The second section also includes a clip 472 that is used to retain a proximal end of the EMD 470. FIG. 48 is a perspective view of an EMD adapter and EMD installed in a cassette in accordance with an embodiment. The first section 466 of the EMD adapter 462 is positioned in the cradle 456 and midsection 458 of the cassette 450. The second section 468 of the END adapter 462 is positioned in the off-axis recess 460. A rapid exchange EMD 470 (e.g., a rapid exchange balloon) is positioned in the second section of the EMD adapter 462 and the proximal end of the EMD 470 is clipped into place using clip 472. The first section 466 of the EMD adapter 462 may be used to receive a guidewire (not shown) from a proximal device module (not shown). The guidewire may pass through the cassette 450 and be driven by the more proximal device module. The EMD adapter 462 provides buckling support for the guidewire. In another embodiment, an EMD adapter may be configured to interface with a balloon guide catheter. For a balloon guide catheter, an EMD adapter may be configured to constrain the proximal end of the balloon guide catheter for linear motion, but not to allow the balloon guide catheter to be rotated.

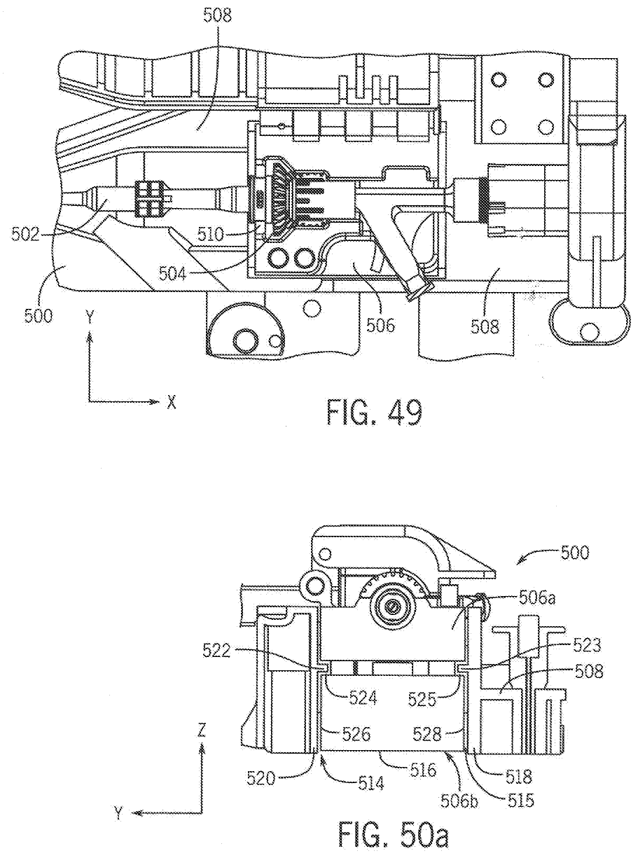

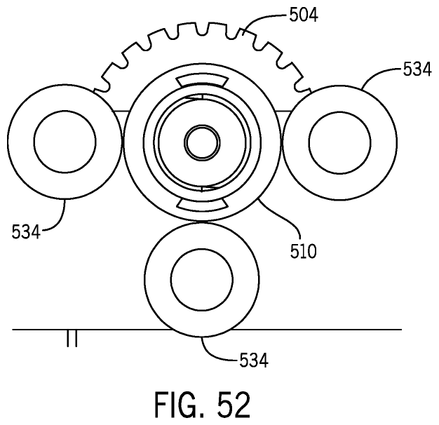

It may be desirable to measure the load applied to an EMD as it is hub driven using a device module in a robotic drive by using a load sensing system. To accurately sense the linear force on an EMD hub, the components in the device module (e.g., the EMD and EMD hub) to be sensed should be isolated from external forces. A device support, as it is tensioned, redirected through the cassette and split, imparts forces on the cassette. The connection of a connector of a device support and a forward constraint of another cassette also imparts forces. In an embodiment, the cassette of a device module may be configured separate the portion of the cassette that supports an EMD from the rest of the cassette to isolate linear forces on the EMD hub. FIG. 49 is a top view of s cassette with a floating (or isolated) interface and a rigid support section in accordance with an embodiment. Cassette 500 includes a floating (or isolated) interface (or component) 506 located in the cassette so as to provide support for an EMD 502 positioned in the floating interface 506. The remainder of the cassette 500 (e.g., the housing) forms a rigid support 508. The EMD 502 includes a rotational drive element 504 (e.g., an on-device adapter such as a gear) configured to interface with the drive mechanisms e.g., a bevel gear (not shown) in the floating interface 506. The rotational drive element 504 is supported in a rotational drive element cradle 510 of the floating interface 506. The floating interface 506 is floating with respect to the rigid support 508 portion of the cassette 500. For example, the floating (or isolated) interface 506 is moveable within and/or relative to the rigid support 508. In an embodiment, the floating interface 506 is isolated from the rigid support such that the floating interface 506 is not fixed to the rigid support 508. As discussed further below, the floating interface 506 is configured to be isolated from loads other that the actual load acting on the EMD 502. The rigid support 508 reacts forces such as, for example, forces from a device support connected to the cassette. To reduce measurement noise for rotational forces, a cradle 510 supporting the rotational drive element 504 (e.g., a gear) of an EMD 502 may be formed from low friction static material. In another embodiment, the cradle 510 may include rollers 534 as shown in FIG. 52. For example, the rollers 534 may be sliding or rolling bearings.

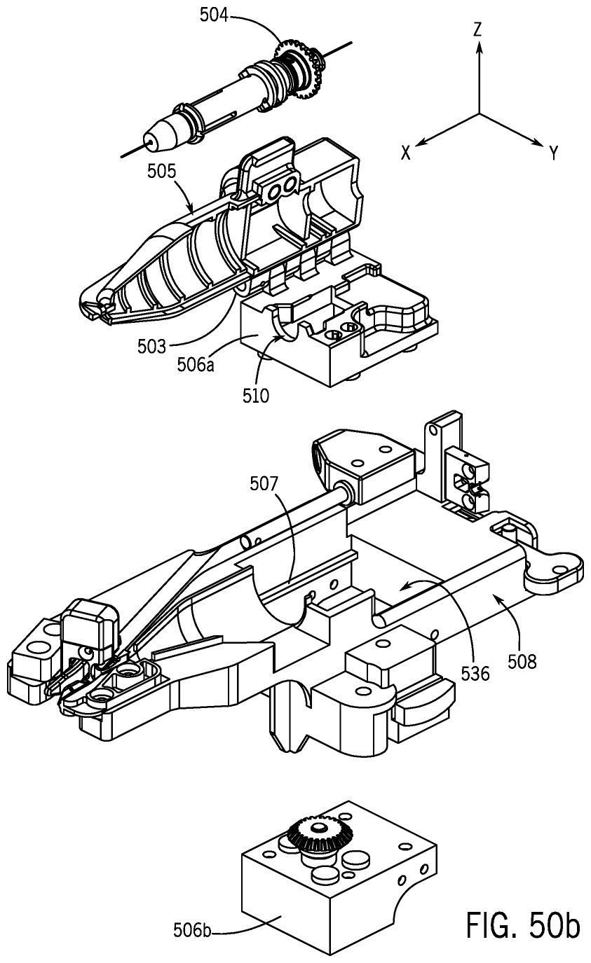

FIG. 50a is an end cross-sectional view of a floating (or isolated) interface and rigid support section of a cassette in accordance with an embodiment. The floating (or isolated) interface 506 is positioned within a recess or opening 536 (shown in FIG. 50b) in the cassette 500 housing and is separated from the rigid support 508 by a first slot 514 and a second slot 515 and confined to a limited range of motion. In an embodiment, the floating interface 506 includes a first component 506a and a second component 506b as discussed further below with respect to FIG. 50b. The floating interface 506 is loosely contained within the recess 536 (shown in FIG. 50b). The range of motion of the floating interface 506 allows the floating interface 506 to be mounted to a drive module (e.g., drive module 310 shown in FIG. 25)), in particular, a load sensing portion of a drive module while giving allowances for tolerances between interfacing components. The first slot 514 and the second slot are configured to allow limited movement of the floating interface 506 in the X and Y directions. The floating interface 506 is also floating (or isolated) but captive in the first slot 514 and the second slot 515 in the z-direction due to a first tab 522 on a first side 518 of the rigid support 508 proximate the first slot and a second tab 523 on a second side 520 of the rigid support 508 proximate the second slot 515. The floating interface 506 includes a first recess 524 on a first side 526 of the floating interface 506 and a second recess 525 on a second side 528 of the floating interface 506. The tabs 522 are loosely positioned in the recesses 524 of the floating interface 506. First tab 522 is loosely positioned on the first recess 524 of floating interface 506 and the second tab 523 is loosely positioned in the second recess 525 of the floating component 506. In one embodiment, the floating interface 506 and the rigid support 508 exist as a single unit, rather than two completely independent pieces which can aid in the usability and setup of a robotic drive. A contactless, frictionless interface between the floating interface 506 and the rigid support 508 is enabled by having the floating interface 506 floating in the z-direction. A contactless interface is achieved when the floating interface 506 is mounted to a drive module (e.g. drive module 310 shown in FIG. 25). For example, the positioning pins 313 (shown in FIG. 25) on the drive module 310 lift the floating interface 506 to a height relative to the rigid support 508 where a contactless interface is achieved as shown in FIG. 50. In one embodiment, the height is 1 mm. In other embodiments, the height is less than 1 mm and in other embodiments the height is greater than 1 mm.

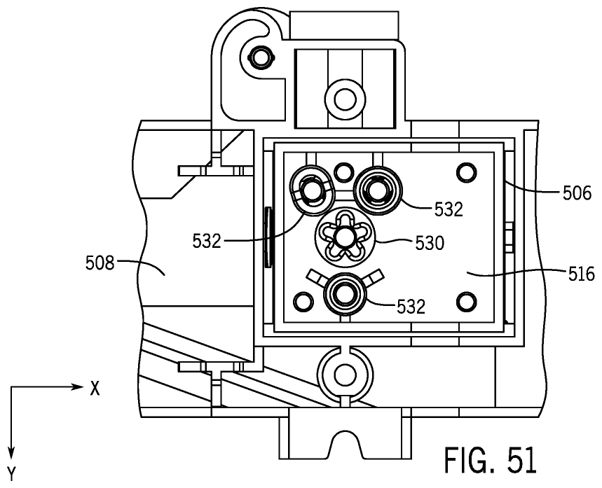

A bottom surface 516 of the floating (or isolated) interface 506 is configured to couple to a drive module. FIG. 51 is a bottom view of the floating interface of a cassette in accordance with an embodiment. The bottom surface 516 of the floating (or isolated) interface 506 includes a connector 530 to receive a coupler (e.g., coupler 314 shown in FIG. 25) of a drive module and connection points 532 configured to receive various types of connection members of the drive module. For example, positioning pins 313 (shown in FIG. 25) may fit into a series of holes and slots in the bottom surface 516 of the floating interface 506. Positioning pins 313 may be used to constrain the floating interface 506 and the drive module in the X and Y directions. In an embodiment, the floating interface 506 may also be constrained in the Z direction by using magnets positioned in one or more connection point 532. In another embodiment, floating interface 506 is constrained in the z direction by friction with the connection points 532. In one embodiment, slots are used to interact with the positioning pins 313 of the drive module to constrain floating interface 506.

As mentioned, the floating (or isolated) interface 506 includes a first component 506a and a second component 506b. FIG. 50b is an exploded isometric view of a cassette showing a first component and a second component of a floating (or isolated) interface in accordance with an embodiment. The first component 506a is placed within a recess 536 of the rigid support portion (or cassette housing) 508 of the cassette in a direction toward a drive module 310 (shown in FIG. 25) when the cassette is in the in-use position secured to the drive module 310. The second component 506b is placed within the recess 536 from a direction away from the drive module 310 toward the first component 560a. The floating (or isolated) interface 506 is positioned within and separate from the rigid support 508 in at least one direction when the floating interface 506 is connected to the drive module. The rigid support (or cassette housing) 508 include two longitudinally oriented rails 507 located within the recess 536. In an embodiment, the rails 507 act as the tabs 522 and 523 (discussed above with respect to FIG. 50a). The first component 506a is located on the top surface of the rails 507 closer to the top surface with the rigid support 508 and the second component 506b is located proximate to the bottom surface e of the rails 507 closest to the drive module (e.g., drive module 310 shown in FIG. 25). Note that although the direction of assembly of the first component 506a and the second component 506b of the floating interface 506 is described in relation to the in-use position, the first and second components 506a, 506b of the floating component 506 are installed away from the drive module. Stated another way, the first component 506a of the floating interface 506 is inserted into the recess 536 in a direction from a top surface of the cassette to the bottom surface of the cassette in a direction generally perpendicular to a longitudinal axis of the cassette housing.

The first component 506a and the second component 506b of the floating interface 506 are secured to one another. In one embodiment, a mechanical fastener or a plurality of fasteners may be used to secure the first component 506 to the second component 506b of the floating interface 506. In other embodiment, the first component 506a and the second component 506b may be secured together using for example, magnets or adhesive. The first component 506a and the second component 506b may be releasably secured to one another or non-releasably secured to one another.

In an in-use position where the second component 506b of the floating interface 506 is releasably secured to a drive module (e.g., drive module 310 shown in FIG. 25), the first component 506a and the second component 506b are spaced from the rails 507 of the rigid support 508 such that the first component 506a and the second component 506b are in a non-contact relationship with the rigid support 508. In one embodiment, the cassette includes a cassette cover 505 pivotally coupled by a hinge 503 to the floating interface 506 separate from and in non-contact with the rigid support 508. For example, the cover 505 may be pivotally coupled by hinge 503 to the first component 506a. In another embodiment, the cover 505 may be connected to the first component 506a by other connection mechanism, such as snap fits.

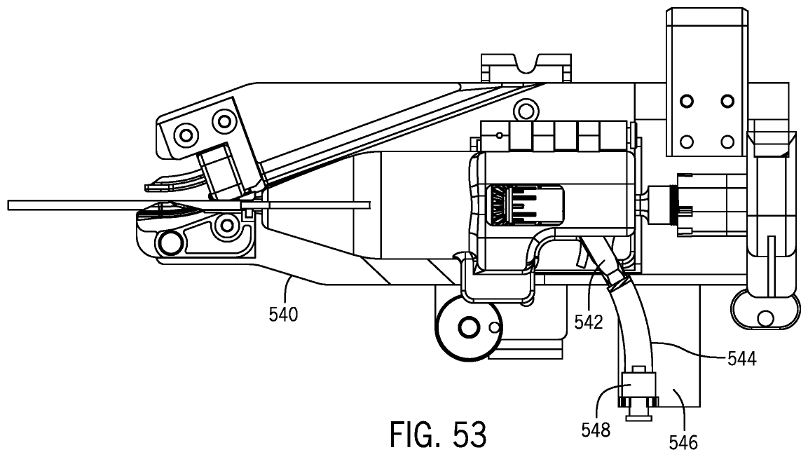

Often, an EMD (e.g., a catheter) in a cassette may be connected via a side port of a hemostasis valve connected to the EMD to various tubing to, for example, supply a saline drip, to allow for contrast injection, to allow for aspiration, etc. In a robotic drive that manipulates EMDs linearly it would be advantageous to account for tubing connections, in particular, to provide a support assembly so that the tubing does not snag or pull on the hemostasis valve. FIG. 53 illustrates a cassette with a support assembly for anchoring tubing and fluid connections in accordance with an embodiment. The support assembly for tubing and fluid connection includes a flexible section of tube 544 attached at one end to a side port 542 of a hemostasis value positioned in a cassette 540. A second end of the flexible section of tube 544 is attached to a clip 548 which is mounted to a support 546. The support 546 is connected to the cassette 540. The second end of the tube 544 and the clip 548 may be configured to provide a connector (e.g., a female port) to be attached to a tube or other fluid connection. The support assembly creates strain relief so that if the tubing 544 were tugged, the force would be reacted by the connection to the support 546 and not the hemostasis valve 542. In another embodiment, the strain relief tube 544 may also terminate in a multi-port stopcock manifold, which would allow for multiple tubing connections to remain in place during a procedure.

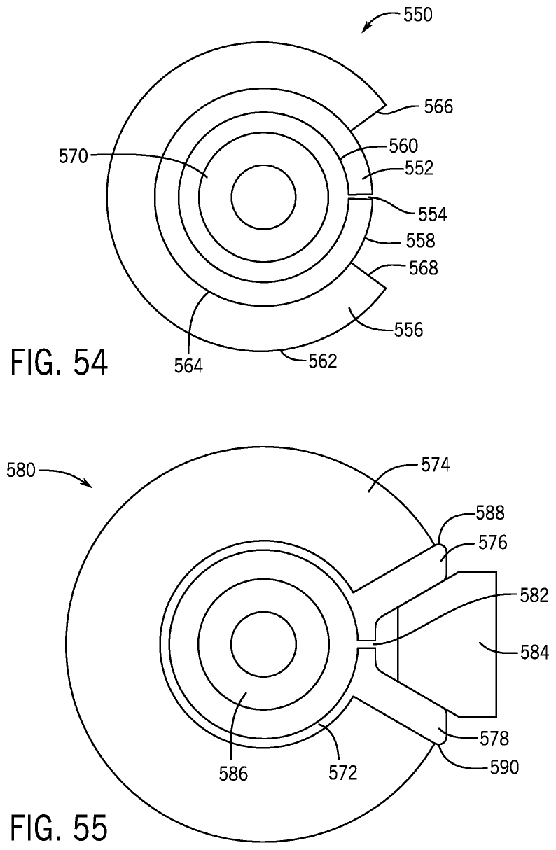

As mentioned above, the profile of a device support formed from a flexible tube with a longitudinal slit should support being opened and closed, for example, to allow EMDs to be loaded into the device support and retained in the device support so as to not pop out and buckle. FIG. 54 is an end cross-sectional view of a device support in accordance with an embodiment. In FIG. 54, a device support 550 includes a first (or inner) flexible tube 552 and a second (or outer) flexible tube 556. The inner tube 552 includes a lengthwise slit 554, an outer diameter 558 and an inner diameter 560. In an embodiment, inner tube 552 is a thin-walled tube to allow the lengthwise slit 554 to be more easily spread apart and closed. The outer tube 556 includes an outer diameter 562 and an inner diameter 564. In addition, the outer tube 556 includes a lengthwise opening defined by a first side 566 and a second side 568. The outer tube 556 is disposed around the outer diameter 558 of the inner tube 552. The outer tube 556 may be formed using a material that provides sufficient force to hold the slit 554 of the inner tube 552 in a “closed” position, for example, so the sides of the slit 554 are in contact and an EMD 570 positioned in the inner tube 552 is retained in the inner tube 552. The material used to form the outer tube 556 should also be configured to allow the slit of the inner tube to be forced apart when a force from, for example, a splitter is applied. In an embodiment, the inner diameter 564 of the outer tube 556 is smaller than the outer diameter 558 of the inner tube 552.

As discussed above, a splitter or wedge may be used to spread apart the lengthwise slit of a device support to allow the device support to encapsulate an EMD. FIG. 55 is an end cross-sectional view of a device support and splitter in accordance with an embodiment. In FIG. 55, a device support 580 includes a first (or inner) flexible tube 572 and a second (or outer) flexible tube 574. The inner tube 572 includes a lengthwise slit 582, a first arm element 576 and a second arm element 578. In an embodiment, inner tube 572 is a thin-walled tube to allow the lengthwise slit 582 to be more easily spread apart and closed. The outer tube 574 includes a lengthwise opening defined by a first side 588 and a second side 590. The outer tube 574 is disposed around an outer diameter of the inner tube 572. The first arm 576 and the second arm 578 of the inner tube 572 are disposed within the opening of the outer tube 574. In the embodiment shown in FIG. 55, the first arm 576 is in contact with the first side of the opening and the second arm 578 is in contact with the second side 590 of the opening. The first 576 and second arm 578 provide a surface that may run over a splitter, for example, splitter 584, as the device support is advanced over the splitter 584 to force apart the slit 582 of the inner tube 572 to encapsulate an EMD 586. The first 576 and second 578 arms prevent the splitter 584 from making contact (e.g., rubbing) with the EMD 586 as the device support 580 is advanced over the splitter 584. Accordingly, the first 576 and second 578 arms may reduce or eliminate friction forces on the EMD 586 which can cause damage to the EMD 586.

Computer-executable instructions for supporting and driving elongated medical devices in a robotic catheter-based procedure system in according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital versatile disks (DVD) or other optical storage, magnetic cassettes, magnetic tape, magnetic disk storage or other magnetic storage devices, or any other medium which can be used to store the desired instructions and which may be accessed by system 10 (shown in FIG. 1), including by internet or other computer network form of access.

A control computing system as described herein may include a processor having a processing circuit. The processor may include a central purpose processor, application specific processors (ASICs), circuits containing one or more processing components, groups of distributed processing components, groups of distributed computers configured for processing, etc. configured to provide the functionality of module or subsystem components discussed herein. Memory units (e.g., memory device, storage device, etc.) are devices for storing data and/or computer code for completing and/or facilitating the various processes described in the present disclosure. Memory units may include volatile memory and/or non-volatile memory. Memory units may include database components, object code components, script components, and/or any other type of information structure for supporting the various activities described in the present disclosure. According to an exemplary embodiment, any distributed and/or local memory device of the past, present, or future may be utilized with the systems and methods of this disclosure. According to an exemplary embodiment, memory units are communicably connected to one or more associated processing circuit. This connection may be via a circuit or any other wired, wireless, or network connection and includes computer code for executing one or more processes described herein. A single memory unit may include a variety of individual memory devices, chips, disks, and/or other storage structures or systems. Module or subsystem components may be computer code (e.g., object code, program code, compiled code, script code, executable code, or any combination thereof) for conducting each module's respective functions.

This written description used examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. The order and sequence of any process or method steps may be varied or re-sequenced according to alternative embodiments.

Many other changes and modifications may be made to the present invention without departing from the spirit thereof. The scope of these and other changes will become apparent from the appended claims.

We claim:

1. An apparatus for providing support to an elongated medical device between a first device module and a second device module coupled to a linear member of a robotic drive for a catheter-based procedure system, the second device module located in a position along the linear member distal to the first device module, the apparatus comprising:
a device support having a distal end and a proximal end, wherein a section of the device support is positioned within the first device module; and
a connector attached to the distal end of the device support, the connector including an attachment mechanism for engaging a proximal end of the second device module;
wherein the proximal end of the device support is configured to be coupled to the second device module.

2. The apparatus according to claim 1, wherein the device support is under tension between the distal end and the proximal end.

3. The apparatus according to claim 1, wherein the device support is configured to move relative to the first device module.

4. The apparatus according to claim 3, wherein the device support is configured to move through a channel of the first device module.

5. The apparatus according to claim 1, wherein the device support is a tube having a lengthwise slit and the connector is configured to hold open the distal end of the device support.

6. The apparatus according to claim 1, wherein the attachment mechanism of the connector is configured to engage a forward constraint on the proximal end of the second device module.

7. The apparatus according to claim 6, wherein the connector attaches to the forward constraint along a longitudinal axis of the elongated medical device positioned within the device support.

8. A cassette for use in a robotic drive of a catheter-based procedure system, the cassette comprising:
a housing having a distal end and a proximal end;
a device support having a lengthwise slit, a distal end and a proximal end, wherein a section of the device support is positioned within the housing;
a connector attached to a distal end of the device support; and
a splitter positioned at the distal end of the housing of the cassette at an entry point for an elongated medical device into the device support;
wherein, in a first position, the connector is located proximal to the entry point and in a second position, the connector is located distal to the entry point.

9. The cassette according to claim 8, wherein the distal end of the housing comprises a recess.

10. The cassette according to claim 9, wherein the first position is a retracted position and in the first position, the connector is located in the recess.

11. The cassette according to claim 9, wherein the splitter is positioned in the recess.

12. The cassette according to claim 11, further comprising a guide positioned in the recess on an opposite side of a path for the device support than the splitter.

13. The cassette according to claim 12, wherein the splitter is configured to hold open the slit of the device support as the connector and the device support are moved over the splitter,

14. The cassette according to claim 13, wherein the splitter is configured to hold open the slit of the device support on both the proximal side of the entry point and a distal side of the entry point.

15. The cassette according to claim 8, wherein the first position is configured to enable loading of the elongated medical device into the device support.

16. The cassette according to claim 8, wherein in the first position the connector is off axis to a longitudinal axis of the elongated medical device.

17. A device support for providing support to an elongated medical device between a first device module and a second device module coupled to a linear member of a robotic drive of a catheter-based procedure system, the device support comprising:
a first tube having a lengthwise slit configured to move between a first position and a second position, the first tube having an inner diameter and an outer diameter; and
a second tube having a lengthwise opening, an inner diameter and an outer diameter, the second tube disposed around the outer diameter of the first tube and configured to provide a force on the first tube to hold the first tube in the first position.

18. The device support according to claim 17, wherein the inner diameter of the outer tube is smaller than the outer diameter of the inner rube.

19. The device support according to claim 17, wherein the inner tube further comprises a first arm and a second arm positioned in the lengthwise opening of the second tube.

20. The device support according to claim 19, wherein the first arm and the second arm are configured to receive a splitter.

21. The device support according to claim 17, wherein the first tube is formed from a flexible material.

22. The device support according to claim 21, wherein the first tube is configured to provide low friction on the elongated medical device.

23. The device support according to claim 17, wherein the second tube is formed from a flexible material.

24. The device support according to claim 20, wherein the splitter is configured to provide a force on the lengthwise slit of the first tube to hold the first tube in the first position.

25. A cassette for use in a robotic drive of a catheter-based procedure system, the cassette comprising:
a housing having a distal end and a proximal end;
an entry point to a device support on the distal end of the housing; and
a modular section of the housing located between the proximal end and the entry point on the distal end, the modular section configured to receive a plurality of different adapters configured to support different elongated medical devices.

26. The cassette according to claim 25, wherein the modular section comprises:
a midsection; and
a recess positioned off-axis from a longitudinal axis of the cassette.

27. The cassette according to claim 25, wherein the elongated medical device is a balloon guide catheter.

28. The cassette according to claim 25, wherein the elongated medical device is a rapid exchange device.

29. An apparatus for providing support for an elongated medical device in a catheter-based procedure system, the apparatus comprising:
a cassette comprising:
a housing having a distal end and a proximal end;
an entry point to a device support on the distal end of the housing; and
a modular section of the housing located between the proximal end and the entry point on the distal end, the modular section comprising a midsection and a recess positioned off-axis from a longitudinal axis of the cassette; and
an elongated medical device adapter comprising:
a first section configured to receive a first elongated medical device; and
a second section configured to receive a second elongated medical device,
wherein the second section is positioned at an angle from a longitudinal axis of the first section;
wherein the first section of the elongated medical device adapter is positioned in the midsection of the modular section and the second section of the elongated medical device adapter is positioned in the recess of the modular section.

30. The apparatus according to claim 29, therein the first elongated medical device is a guidewire.

31. The apparatus according to claim 29, wherein the second elongated medical device is a balloon guide catheter,

32. The apparatus according to claim 29, wherein the second elongated medical device is a rapid exchange device.

33. The apparatus according to claim 29, wherein the second section of the elongated medical device adapted includes a clip configured to retain a proximal end of second elongated medical device.

34. The apparatus according to claim 29, wherein the elongated medical device adapter further comprises a lid.

35. A cassette for use in a robotic drive of a catheter-based procedure system, the cassette comprising:
a rigid support including an opening; and
an isolated interface positioned within the opening, the isolated interface including a cradle for an elongated medical device;
wherein the recess and the isolated interface are configured to allow a limited range of motion of the isolated interface in the x, y, and z directions relative to the rigid support.

36. The cassette according to claim 35, wherein the isolated interface is isolated from the rigid support and isolates a hub of the elongated medical device from linear forces.

37. The cassette according to claim 35, wherein the rigid support is configured to react forces on the cassette.

38. The cassette according to claim 35, wherein the recess includes a first tab on a first side of the opening and a second tab on a second side of the opening.

39. The cassette according to claim 38, wherein the isolated interface includes a first recess on a first side of the isolated interface and a second recess on a second side of the interface portion.

40. The cassette according to claim 39, wherein the first tab is positioned in the first recess and the second tab is positioned in the second recess.

41. The cassette according to claim 35, wherein the isolated interface includes a bottom surface comprising a connector configured to receive a coupler of a drive module.

42. The cassette according to claim 41, wherein the bottom surface further comprises a plurality of connection points configured to receive connection members of the drive module.

43. The cassette according to claim 42, wherein the connection members when positioned in the plurality of connections points are configured to constrain the isolated interface.

44. The cassette according to claim 42, wherein at least one of the plurality of connection points includes a magnet.

45. The cassette according to claim 38, wherein the first tab is a rail and the second tab is a rail.

46. The cassette according to claim 35, wherein the isolated interface comprises a first component and a second component.

47. The cassette according to claim 45, wherein the isolated interface comprises:
a first component disposed on a top surface of the first tab and the second tab; and
a second component disposed proximate to a bottom surface of the first tab and the second tab.

48. A cassette for use in a robotic drive of a catheter-based procedure system, the cassette comprising:
a rigid support portion;
an interface portion configured to support a hemostasis valve having a port; and
an apparatus for anchoring a fluid connection to the hemostasis valve comprising:
a flexible tube having a first end and a second end, the first end of the flexible tube configured to connect to the port of the hemostasis valve; and
a clip attached to the rigid support portion and the second end of the flexible tube.

49. The cassette according to claim 48, wherein the second end of the flexible tube is configured to provide a connector to the fluid connection.

50. The cassette according to claim 48, wherein the second end of the flexible tube includes a multi-port connector.

51. The cassette according to claim 48, wherein the apparatus for anchoring a fluid connection is configured to provide strain relief.
